(19) United States
(12) Patent Application Publication
(10) Pub. No.: US 2008/0194798 A1
(43) Pub. Date: Aug. 14, 2008

(54) Novel Iminecalixarene Derivatives, Method for Preparing the Same, Self-Assembled Monolayer Prepared by Using the Iminecalixarene Derivatives or Aminocalixarene, and Protein Chip Using the Self-Assembled Monolayer

(76) Inventors: Tae Sun Kim, Gangwon-do (KR); Keum Soo Song, Gangwon-do (KR); Jung Hoon Kim, Gangwon-do (KR)

Correspondence Address: HOWREY LLP, C/O IP DOCKETING DEPARTMENT, 2941 FAIRVIEW PARK DRIVE, SUITE 200, FALLS CHURCH, VA, 22042-2924, US

(21) Appl. No.: 11/910,164

(22) PCT Filed: Jun. 14, 2006
(86) PCT No.: PCT/KR2006/002258

§ 371 Date: Nov. 28, 2007

(30) Foreign Application Priority Data
Nov. 1, 2005 (KR) 10-2005-0103857
Nov. 18, 2005 (KR) 10-2005-0110824

(51) Int. Cl. C07K 16/00; C07C 211/40

(52) U.S. Cl. 530/387.1; 564/305

(57) ABSTRACT

The present invention relates to iminecalixarene derivatives, iminecalixarene derivative monolayers prepared as a monolayer on a solid substrate by using said iminecalixarene derivatives, a protein chip substrate prepared by fixing all kinds of proteins on a chip substrate in a solution phase without any treatment, while ammonium groups on the surface of the proteins are irreversibly recognized by said iminecalixarene derivative monolayer, and protein chips prepared by using the protein fixation on said substrate.

### TECHNICAL FIELD

The present invention relates to novel tetraiminecalixarene derivatives, method of preparation thereof, self-assembled monolayer prepared by using them, unmodified protein fixation method using ionic recognition on the self-assembled monolayer, and protein chip preparation technology using it. More particularly, the present invention relates to tetraiminecalixarene derivatives capable of fixing protein such as antigen, antibody, etc., or compounds having an ammonium functional group, etc. on the surface of a solid substrate by molecular recognition, a method of preparation thereof, a method for preparing it in a monolayer on a solid substrate such as a glass substrate or a gold substrate, and a method for preparing a protein monolayer, i.e., protein chip using a self-assembled monolayer thereof.

Also, the present invention relates to a self-assembled monolayer prepared by using aminocalixarene derivatives, a method for fixing protein thereon in a monolayer by using ionic or molecular recognition, and a protein chip prepared by using the method. More particularly, the present invention relates to a aminocalixarene derivative monolayer prepared in a monolayer on a solid substrate such as a glass substrate or a gold substrate by using the compound of formula 1 or formula 2, a method for fixing protein such as antigen, antibody, etc., especially protein having the molecular weight of 10 kD or more, on the surface of the monolayer at high density by molecular recognition, and a protein chip prepared by said fixation of protein.

### BACKGROUND ART

Development and differentiation of vital phenomenon is controlled by functional interaction such as protein-protein, protein-ligand(or agent), antigen-antibody, enzyme-substrate, etc. Thus, the research to discover the function or role of the biomolecule that uniquely interacts with a specific protein or ligand is becoming a main project in the fields of life science, health, medical treatment, etc. In order to fix such various proteins on a single solid substrate, i.e. a chip and measure interaction of protein-protein or protein-ligand, the protein chip wherein a number of proteins are fixed is being researched actively. Particularly, the method of fixing enzyme, antigen, antibody, etc. on a solid substrate is the most primary infratechnology in life science such as immunochemistry, enzymochemistry, etc. or in various fields wherein proteins are used, such as a diagnosis kit diagnosing diseases by using proteins. For example, the Enzyme-Linked Immunoassay, EIA, a method most commonly used for diagnosis, is a diagnosis method that antibody is physically absorbed in the bottom of 96-microtiter well made of plastics or antibody is fixed by a chemical bonding method that reacts various functional groups existing at the surface of the antibody to prepare a product, and it is reacted with the antigen of blood serum and developed in color using the secondary antibody-enzyme conjugate, and then the concentration of antigen is measured through fluorescence analysis in order to analyze a certain protein or a specific protein that becomes a disease cause. Various disease diagnosis kits prepared by this method are on the market.

Besides physical absorption or chemical bonding method, a protein fixation method that fixes protein such as antibody wherein biotin is attached by biotin-streptavidin molecular recognition after fixing a protein named streptavidin on a solid substrate wherein biotin is attached by hydrogen bonding is known.

The problems occurring in protein fixation technology such as conventional physical absorption method and chemical bonding method, and the method using biotin-streptavidin molecular recognition, which currently started being used, etc. are as follows.

1. Fixation Density: Although this is the field that more research has proceeded than any other among protein fixation technology, for various kinds of proteins currently being used for fixation, solid substrates capable of preparing a protein monolayer which completely covers the whole space upon fixation have rarely been published. Unless the density of protein fixed on the surface reaches the density that can make a perfect level of a monolayer (in case of antibody, it is 1.1-1.4 μg/cm²), the following problems can occur: that specific antigen is fixed not on the antibody but nonspecifically on the surface wherein solid substrate is directly exposed; that since the amount of fixed antibody is small, the amount of combined antigen becomes small, as a result causing difficulty in reading; and that the concentration of readable protein is not distinguished from conventional technology.

2. Reproducibility: It is the most important technology to put a product on the market in the protein fixation technology. When several to dozens of kinds of proteins are fixed, the precise diagnosis results can be obtained only when fixed protein achieves the same levels of fixation density and activity. Protein fixation technology currently used include the chemical bonding on a solid substrate, the biotin-streptavidin method, or a method using polymers that can make the maximum chemical bonding on a glass slide, such as polymers of the polylysine series. However, since most of them require reaction between protein and a specific functional group and fixation occurs after the reaction, the conventional protein fixation technology have many problems in maintaining the same results, i.e., reproducibility when several incomplete reactions are carried out.

3. Superhigh speed fixation: Upon fixation, the fixation occurs on the surface of a solid substrate in a solution phase, and if protein is dissolved in a solution phase, the activity of protein in a solution phase tends to decrease as time passes by. However, since the activity hardly decreases after fixation, a solid substrate capable of superhigh speed fixation and completing fixation within one hour needs to be developed in order to minimize the activity reduction upon fixation, but it has not been developed yet.

4. Unmodified protein Fixation: Even if the protein fixation density, i.e., the reproducibility where the number of proteins to be fixed are maintained at the same level within a certain range, is secured and if the same number of proteins is fixed, in order to carry out fixation maintaining the activity of protein at the same level within a certain range of error, the fixation should take place in a controllable solution phase and to achieve it, the development of technology that does not require a chemical bonding process which can affect the protein activity and fixes unmodified protein directly in a solution phase is necessary. Since, except the technology using crown compounds that the present researchers published several years ago, no unmodified protein fixation technology has been developed yet, unmodified protein fixation technology that does not involve attachment of a functional group and the development of the solid substrate wherein the technology can be applied is necessary.

5. Technology for an analysis of a trace protein

Since the concentration of antibody protein, etc. which can be analyzed by using conventional protein fixation technology remains at the level of several ng/ml, a diagnosis product capable of quantitative analysis of protein existing at a concentration lower than that has not been developed.

If a technology capable of quantitatively analyzing protein existing even at a concentration of several μg/ml or lower is developed, new cancer identification protein which couldn't be analyzed with conventional technology can be discovered, and at the same time, by making possible the quantitative analysis even when the concentration of cancer identification protein is very low, the development of new concept diagnosis protein that makes possible early diagnosis and disease prevention is expected to be possible; however, it has not been developed until now.

6. Long-Term Storage Technology

Protein loses its activity significantly when stored over a long period of about 3-6 months, and since the reduction ratio is not constant, it causes many problems with its commercialization. In order to obtain the same diagnosis results even upon such long storage, the Protein Chip Research Association, etc. has suggested the need for development of the technology minimizing the phenomenon that the protein fixed on a solid substrate by nonspecific attraction is attracted to the surface, which happens when protein is fixed at a low density or by physical absorption, by fixing protein at high density without empty space, that is, the technology to minimize the protein activity reduction caused by alteration of an active site, which happens when it is difficult to maintain the shape of protein, that is, the protein chip preparation technology capable of high density fixation without leaving empty space.

### DISCLOSURE

### [Technical Problem]

In order to solve the problems of conventional protein fixation technology, the density of proteins fixed on the surface of a solid substrate must be high enough to form a perfect level of a monolayer, and the fixation density and the activity of the proteins must be maintained at the same level, and superhigh speed fixation must be possible to reduce activity reduction upon fixation.

### [Technical Solution]

An objective of the present invention is, in order to solve product reproducibility problems that have hindered protein chips from being put on the market among the above-mentioned problems of various conventional protein fixation methods, to provide high density fixation technology which do not leave empty space for obtaining homogeneous fixation density and maintaining the same level of activity. In addition, another objective of the present invention is to provide technology that solves the problems of the irregularity of reading results, which happens because the protein activity decreases in a solution phase as time passes by and thus the protein activity after fixation becomes irregular.

In addition, considering that the active site, which contributes to protein activity, is maintained mainly by hydrogen bonding, another objective of the present invention is also to provide iminecalixarene derivatives essential for developing reproducible protein chips by solving the problems of activity irregularity caused by the alteration of active sites that happens by the chemical bond stronger than hydrogen bond upon chemical bonding and the preparation method thereof.

In addition, another objective of the present invention is, by attaching said compounds to a glass substrate (amine slide glass) wherein amine functional groups are attached, siliconwafer, fused silica, or gold substrate, etc., to provide an iminecalixarene derivative monolayer or an aminocalixarene derivative monolayer wherein all kinds of proteins are fixed in a solution phase without any treatment at superhigh speed in a short time of 30 minutes to 1 hour to prepare a protein monolayer.

In addition, another objective of the present invention is to provide a technology for preparing a protein chip, i.e. a protein monolayer prepared using an iminecalixarene derivative monolayer or an aminocalixarene derivative monolayer wherein all kinds of proteins are fixed at high density and superhigh speed without any alteration.

### ADVANTAGEOUS EFFECTS

Distinguished completely from the conventional method of preparing protein chip by fixing protein using conventional chemical bonding, physical absorption, or biotin-streptavidin, which is being generally used over the world, the present invention is a novel technology wherein once unmodified protein is coated on an imincalixarene derivative monolayer or an aminocalixarene derivative monolayer, protein is fixed on the surface of the monolayer irreversibly at superhigh speed through ionic/molecular recognition and at the same time with the maximum density fixation. Also, the present invention relates to a new protein chip preparation technology that not only keeps the amount of fixed proteins, i.e., the density, homogeneous by fixing proteins at the maximum level all the time, but also obtains high reproducibility by maintaining the same level of protein activity through superhigh speed fixation.

The protein monolayer prepared by using the novel iminecalixarene derivatives or the aminocalixarene derivatives developed according to the present invention is a novel technology that can solve the problems of heterogeneousness of fixation density and the activity reduction that occur in conventional protein fixation methods, and the problems of irregularity of an fixed protein activity, which is caused by the activity reduction of proteins left in a solution phase for a long time due to slow fixation technology. In addition, the active site that contributes to protein activity is mainly maintained by hydrogen bonds, and the present invention is a novel technology that, by using molecular recognition, can solve problems that the activity of fixed proteins is irregularly reduced since the alteration of the active site occurs by the chemical bond with a solid substrate stronger than the hydrogen bonds when chemical bonding is carried out using an aldehyde chip, etc.

In addition, the present invention provides a protein chip preparation technology that completely solves the reproducibility problem of the protein chips, which has been the biggest obstacle to putting protein chips on the market, by providing the preparation technology of an iminecalixarene derivative monolayer for protein fixation or an aminocalixarene derivative monolayer, novel iminecalixarene derivatives, and the preparation method thereof.

In addition, the present invention provides an iminecalixarene derivative monolayer or an aminocalixarene derivative monolayer that can make a protein monolayer by fixing all kinds of proteins, regardless of molecular weight, on a solid substrate in a solution phase in a short time of 30 minutes to 1 hour at superhigh speed without any treatment by attaching iminecalixarene derivatives of the formula 2 or 4 to glass substrate (amine slide glass) wherein amine functional groups are attached, siliconwafer, fused silica, etc., or attaching iminecalixarene thiol derivatives of the formula 2 or 4 to a gold substrate, or attaching aminocalixarene derivatives of the formula 6 or 7 to a glass substrate (amine slide glass) wherein amine, alcohol, or thiol functional groups are attached, siliconwafer, fused silica, or a gold substate.

In addition, the present invention provides protein chip preparation technology that can fix proteins of various sizes and shapes having a molecular weight of 1000 (10 kD) or more at an optimal condition with the maximum activity, by providing the world s first iminecalixarene derivative monolayer for protein fixation capable of controlling an fixation speed, increasing fixation speed through the raise of an ionic concentration as in the fixation according to an ion concentration presented in FIG. 9 or FIG. 10 or a competitive reaction of FIG. 14 or FIG. 15, or controlling fixation speed to an optimal state by adding some amine compounds.

In addition, the present invention provides protein chip preparation technology that can simplify protein chip preparation processes and prepare protein chips at a low cost since it can prepare protein chips at superhigh speed with only trace proteins, around 3 times the amount of protein needed to achieve the maximum density.

Particularly, according to the fluorescence analysis results of FIG. 9 and FIG. 11, the present invention provides a technology for preparing protein chips for diagnosis that achieve the maximum sensitivity by enabling the fixed proteins to maintain the theoretically maximum density and maintain the activity of proteins that are combined with antigens at a high level. Said technology will be applied to the development of new technology for preparing various kinds of protein chips, antibody chips for diagnosis, biochips, etc.

### DESCRIPTION OF DRAWINGS

FIG. 1 is a diagram of the process of preparing an amine slide glass according to the method published in a paper (Langmuir, 1996, Vol. 12, pp 5338-5342)

FIG. 2 is a diagram of the self-assembled iminecalixarene derivative monolayer prepared by chemical bonding between the prepared amine slide glass and the iminecalixarene derivatives of the present invention.

FIG. 3 is a diagram of the self-assembled aminocalixarene derivative monolayer prepared by chemical bonding between the prepared amine slide glass and the aminecalixarene derivatives.

FIG. 4 is a diagram showing the preparation process of a self-assembled monolayer which the iminecalixarene derivative to which is attached a thiol functional group among the derivatives of formula 2 and 4 forms on a gold substrate.

FIG. 5 is a diagram showing the preparation process of a self-assembled monolayer which the aminocalixarene derivative to which is attached a thiol functional group among the derivatives of formula 6 and 7 forms on a gold substrate.

FIG. 6 is a diagram showing the process of preparing protein chips wherein when a protein solution is coated on the surface of a solid substrate such as gold or glass slide, covered with a iminecalixarene derivative monolayer according to the present invention, protein is fixed on the surface of the substrate by voluntary molecular recognition at a high density without leaving any space, which leads to the formation of a protein monolayer, i.e. a protein chip.

FIG. 7 is a diagram showing the protein chip according to the present invention, which shows the new technology wherein when an unmodified protein solution that did not go through any chemical change is coated on the surface of a solid substrate such as gold or glass slide, where aminocalixarene derivatives are self-assembled as a monolayer, protein is voluntarily fixed on the surface of the substrate by ammonium ion recognition and molecular recognition of functional groups that can make a hydrogen bonding, such as a guanidine group, which leads to a protein monolayer where proteins are fixed at the theoretically maximum density without leaving empty space, i.e. a protein chip. Actual experiment results are as shown in FIG. 10.

FIG. 8 is a diagram showing that during the preparation of a protein monolayer, that is, a protein chip, by protein fixation according to the present invention, when a terminal which can form a hydrogen bond such as alginine or an ammonium group of a protein is recognized by the aminocalixarene derivative in fixing a protein, the protein fixation occurs with the remaining empty space filled with cations such as ammonium ions.

FIG. 9 shows measurement results that show that in the antibody protein fixation according to the example 15, the kind of ions such as Na⁺, K⁺, NH₄⁺, etc. and the ion concentration affect the protein fixation speed, and that the present invention is a novel technology that can prepare a protein chip by fixing proteins in a short time of 10 to 30 minutes at a maximum density and superhigh speed in the presence of 100-400 mM of NH₄⁺ ion. The density of the fixed proteins was confirmed by conjugation of the protein and fluorescent attached antigen, and it is the same as fluorescence activity of the theoretically maximum density shown in FIG. 11, which shows that the protein fixation in the present invention achieves a maximum density.

FIG. 10 shows actual experiment results that in antibody protein fixation according to example 16, the concentration of NH₄⁺ ion affects protein fixation speed, which shows that the present invention is a novel technology that can prepare a protein chip by fixing proteins in a short time of 10 to 30 minutes at a maximum density and a superhigh speed in the presence of 200-400 mM of NH₄⁺ ion. The density of the fixed protein was confirmed by conjugation of the protein and fluorescent attached antigen, and the results also show that the protein fixation in the present invention is performed near the theoretically maximum density, compared with the fluorescence sensitivity of the theoretically maximum density shown in FIG. 11.

FIG. 11 is the fluorescence sensitivity measured after coating the fluorescent attached antigen 0.5, 1, and 2 times the amount of that combined with protein in the maximum density, and drying it, in order to compare the analysis result of the voluntary protein fixation shown in the FIG. 9 and FIG. 10 with the result of the theoretically maximum density of fixed protein. Among them, the fluorescence sensitivity in case of 1 time is the same as the result obtained at 30 minutes after using an ammonium ion in FIGS. 9 and 10, which confirms that protein fixation of superhigh speed and high density has proceeded.

FIG. 12 is a diagram showing that when a competitive reaction of amine compounds having an amine functional group with proteins occurs, the concentration of the amine compounds affects protein fixation, in order to show that in the protein fixation on the self-assembled aminocalixarene derivative monolayer, amines, i.e., ammonium ions, which exist in the terminal groups, such as lysine, on the protein surface, and ammonium ions attached to the N-terminal of the protein, i.e. amine terminal, etc. are ion recognized irreversibly, which leads to the protein fixation.

FIG. 13 is a diagram showing that when a competitive reaction of imine compounds having an imine functional group with proteins occurs, the concentration of the imine compounds affects protein fixation, in order to show that in the protein fixation on the self-assembled iminecalixarene derivative monolayer, amines, i.e., ammonium ions, which exist in the terminal groups, such as lysine, on the protein surface, and ammonium ions attached to the N-terminal of the protein, i.e. amine terminal, etc. are ion recognized irreversibly, which leads to the protein fixation.

FIG. 14 is the results of a competitive reaction according to the example 19, which show that in the protein fixation on the iminecalixarene derivative monolayer of the present invention, compounds having ammonium groups such as alanine, phenyl alanine, and phenetyl amine are ion recognized in competition with proteins which are being fixed, which decreases actual protein fixation speed.

FIG. 15 is the results of an experiment carried out according to the example 20, which show that when proteins are fixed on the self-assembled aminocalixarene derivative monolayer of the present invention, while compounds having ammonium groups such as lysine, benzyl amine are ion recognized in competition with proteins which are being fixed, thus decreasing actual protein fixation speed, aromatic amines existing in a state of an amine not an ammonium ion in a buffer solution, such as aniline, are not recognized by aminocalixarenes, thus not causing a competitive reaction. Protein fixation also decreases when a competitive reaction is carried out using guanidine groups attached to the terminal of arginine, which easily forms a hydrogen bond. Said results show that in the protein monolayer preparation technology according to the present invention, unmodified proteins are fixed on an aminocalixarene derivative monolayer (protein chip substrate) with ion/molecule recognition by ammonium ions, hydrogen bonds, etc. occurring simultaneously, thus forming a protein monolayer.

FIG. 16 is a diagram that shows attaching Cy-5 fluorescence (Telechem, U.S.A.) to an antigen protein that combines with an antibody protein for fluorescence reading.

FIG. 17 is a diagram showing the state wherein iminecalixarene derivatives of the present invention have recognized four amine compounds (ammonium ions in a solution phase). The spheres existing between ammoniums are anions.

FIG. 18 is the NMR results showing that four amine compounds in the form of ammoniums were actually recognized by iminecalixarene derivatives in a solution phase. The NMR results show that four imine hydrogens in the iminecalixarene derivatives moved to the same position and eight hydrogens in the calixarenes also moved about 1 ppm, which shows that the ammonium ion recognition is irreversible not in a reversible equilibrium. In addition, the results show that amine compounds having a benzene ring are recognized tens of times faster than those without a benzene ring, which supports the competitive reaction results of FIG. 13.

FIG. 19 is the NMR result measuring the molecular recognition of allylamines by an iminecalixarene derivative developed according to the present invention. They are the experiment results of the example 23 showing that 8 hydrogens of CH₂ in an allylamine moved 0.8 ppm and about 4 hydrogens among those attached to double bonds moved, which proves four allylamines are irreversibly recognized by one iminecalixarene. Hydrogens in an iminecalixarene show a moving pattern similar to that of the case when the aminecalixarene derivative of FIG. 18 is recognized, which shows that the ammonium ion recognition of the same pattern has occurred.

### BEST MODE

The novel iminecalixarene derivative essential for preparation of a self-assembled monolayer wherein proteins are fixed in 30 minutes to 1 hour at a superhigh speed without having any treatment has a structure of the following formula 1, 2, 3, or 4.

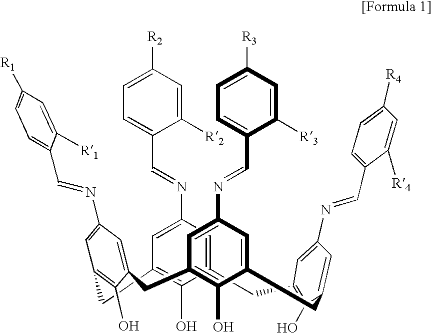

(wherein, R₁, R′₁, R₂, R′₂, R₃, R′₃, R₄, and R′₄ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H₅, —OC₆H₅, —OC₆H₄CH₃, —OC₆H₄C(CH₃)₃, —OC₆H₄CF₃, —OC₆H₄Cl, —OCOCH₃, —NHCOCH₃, —CONHCH₃, —CN, COOH, and —COOR, wherein R represents —CH₃ or —C₂H₅).

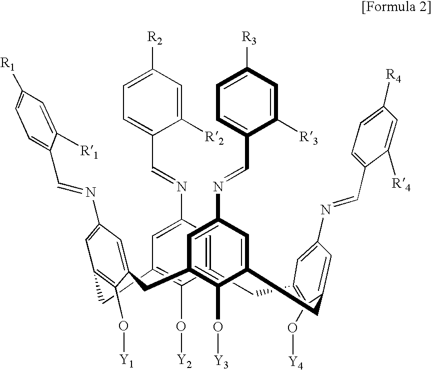

(wherein, R₁, R′₁, R₂, R′₂, R₃, R′₃, R₄, and R′₄ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H₅, —OC₆H₅, —OC₆H₄CH₃, —OC₆H₄C(CH₃)₃, —OC₆H₄CF₃, —OC₆H₄Cl, —OCOCH₃, —NHCOCH₃, —CONHCH₃, —CN, COOH, and —COOR, wherein R represents —CH₃ or —C₂H₅. Also, said Y₁, Y₂, Y₃ and Y₄ are independently selected from the group consisting of —H, —(CH₂)ₙ—CH═O, —(CH₂)ₙ—SH, —(CH₂CH₂O)ₘ—CH₂CH₂—CH═O, —(CH₂CH₂O)ₘ—CH₂CH₂—SH, —(CH₂)ₘ—C₆H₄—(CH₂)_{c}-Z and —CO—(CH₂)ₘ₋₁—C₆H₄—(CH₂)_{c}-Z (wherein, n=2˜15, m=1˜10, c=0˜10, Z=—SH, —CHO, —COOH or —NH₂, and —C₆H₄ and —C₆H₅ are defined as phenyl group.).

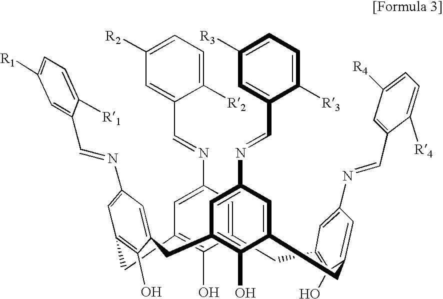

(wherein, R₁, R′₁, R₂, R′₂, R₃, R′₃, R₄, and R′₄ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H₅, —OC₆H₅, —OC₆H₄CH₃, —OC₆H₄C(CH₃)₃, —OC₆H₄CF₃, —OC₆H₄Cl, —OCOCH₃, —NHCOCH₃, —CONHCH₃, —CN, COOH, and —COOR, wherein R represents —CH₃ or —C₂H₅)

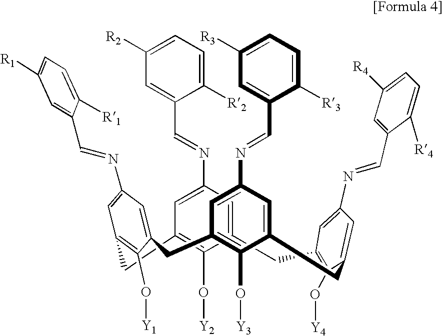

(wherein, R₁, R′₁, R₂, R′₂, R₃, R′₃, R₄, and R′₄ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H₅, —OC₆H₅, —OC₆H₄CH₃, —OC₆H₄C(CH₃)₃, —OC₆H₄CF₃, —OC₆H₄Cl, —OCOCH₃, —NHCOCH₃, —CONHCH₃, —CN, COOH, and —COOR, wherein R represents —CH₃ or —C₂H₅. Also, said Y₁, Y₂, Y₃ and Y₄ are independently selected from the group consisting of —H, —(CH₂)ₙ—CH═O, —(CH₂)ₙ—SH, —(CH₂CH₂O)ₘ—CH₂CH₂—CH═O, —(CH₂CH₂O)ₘ—CH₂CH₂—SH, —(CH₂)ₘ—C₆H₄—(CH₂)_{c}-Z and —CO—(CH₂)ₘ₋₁—C₆H₄—(CH₂)_{c}-Z (wherein, n=2˜15, m=1˜10, c=0˜10, Z=—SH, —CHO, —COOH or —NH₂, and —C₆H₄ and —C₆H₅ are defined as phenyl group.).

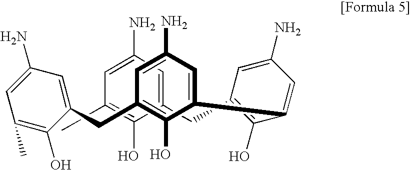

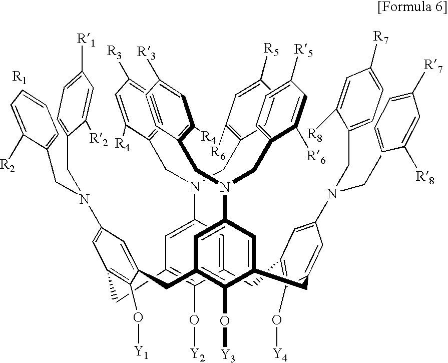

(wherein, R₁, R₂, R₃, R₄, R₅, R₆, R₇, R₈, R₁, R₂, R₃, R₄, R₅, R₆, R₇ and R₈ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H₅, —OC₆H₅, —OC₆H₄CH₃, —OC₆H₄C(CH₃)₃, —OC₆H₄CF₃, —OC₆H₄Cl, —OCOCH₃, —NHCOCH₃, —CONHCH₃, —CN, COOH, and —COOR, wherein R represents —CH₃ or —C₂H₅. Also, said Y₁, Y₂, Y₃ and Y₄ are independently selected from the group consisting of —H, —(CH₂)ₙ—CH═O, —(CH₂)ₙ—SH, —(CH₂CH₂O)ₘ—CH₂CH₂—CH═O, —(CH₂CH₂O)ₘ—CH₂CH₂—SH, —(CH₂)ₘ—C₆H₄—(CH₂)_{c}-Z and —CO—(CH₂)ₘ₋₁—C₆H₄—(CH₂)_{c}-Z (wherein, n=2˜15, m=1˜10, c=0˜10, Z=—SH, —CHO, —COOH or —NH₂, and —C₆H₄ and —C₆H₅ are defined as phenyl group.).

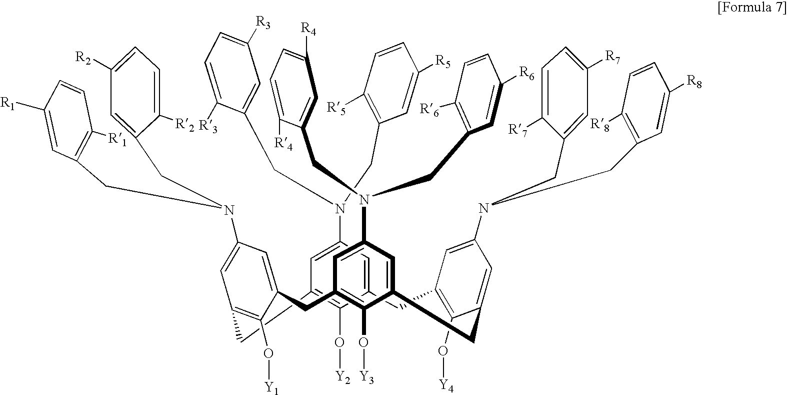

(wherein, R₁, R₂, R₃, R₄, R₅, R₆, R₇, R₈, R₁, R₂, R₃, R₄, R₅, R₆, R₇ and R₈ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H₅, —OC₆H₅, —OC₆H₄CH₃, —OC₆H₄C(CH₃)₃, —OC₆H₄CF₃, —OC₆H₄Cl, —OCOCH₃, —NHCOCH₃, —CONHCH₃, —CN, COOH, and —COOR, wherein R represents —CH₃ or —C₂H₅. Also, said Y₁, Y₂, Y₃ and Y₄ are independently selected from the group consisting of —H, —(CH₂)ₙ—CH═O, —(CH₂)ₙ—SH, —(CH₂CH₂O)ₘ—CH₂CH₂—CH═O, —(CH₂CH₂O)ₘ—CH₂CH₂—SH, —(CH₂)ₘ—C₆H₄—(CH₂)_{c}-Z and —CO—(CH₂)ₘ₋₁—C₆H₄—(CH₂)_{c}-Z (wherein, n=2˜15, m=1˜10, c=0˜10, Z=—SH, —CHO, —COOH or —NH₂, and —C₆H₄ and —C₆H₅ are defined as phenyl group.).

The compounds of the formula 6 and 7 of the present invention can be synthesized using the compound of the formula 5 as a starting material.

The iminecalixarene derivative of the formula 1 is synthesized by using a compound of the formula 5 synthesized according to the example 2 as a starting material and reacting the amine functional group of the formula 5 with a suitable aromatic aldehyde.

The iminecalixarene derivative of the formula 2 is synthesized by reacting a compound having an aldehye, thiol, or carboxyl functional group, etc. at the terminal with —OH, i.e. alcohol functional groups, which is attached to the calixarene of the compound of the formula 1, through ether or ester bond, while alkyl groups having aldehyde or thiol at the terminal are attached to each —OH independently.

The iminecalixarene derivative of the formula 3 is synthesized by using a compound of the formula 5 synthesized according to the example 6 as a starting material and reacting the amine functional group of the formula 5 with a suitable aromatic aldehyde.

The iminecalixarene derivative of the formula 4 is synthesized by reacting a compound having an aldehye, thiol, or carboxyl functional group, etc. at the terminal with —OH, i.e. alcohol functional groups, which is attached to the calixarene of the compound of the formula 3, through ether or ester bond, while alkyl groups having aldehyde or thiol at the terminal are attached to each —OH independently.

In addition, the present invention provides a technology of preparing a oligo-DNA chip, i.e. an oligo-DNA monolayer wherein all kinds of oligo-DNAs are fixed at a high density by attaching said compound of the formula 2 to gold substrate or glass substrate wherein amine groups are attached.

In addition, the present invention provides a technology of preparing an aminocalixarene derivative monolayer by attaching the compound of said formula 6 or 7 to a gold substrate or a solid substrate such as a glass, siliconwafer, fused silica, wherein functional groups that can form chemical bonds are attached, and a protein chip, i.e. a protein monolayer wherein all kinds of proteins are fixed at a high density.

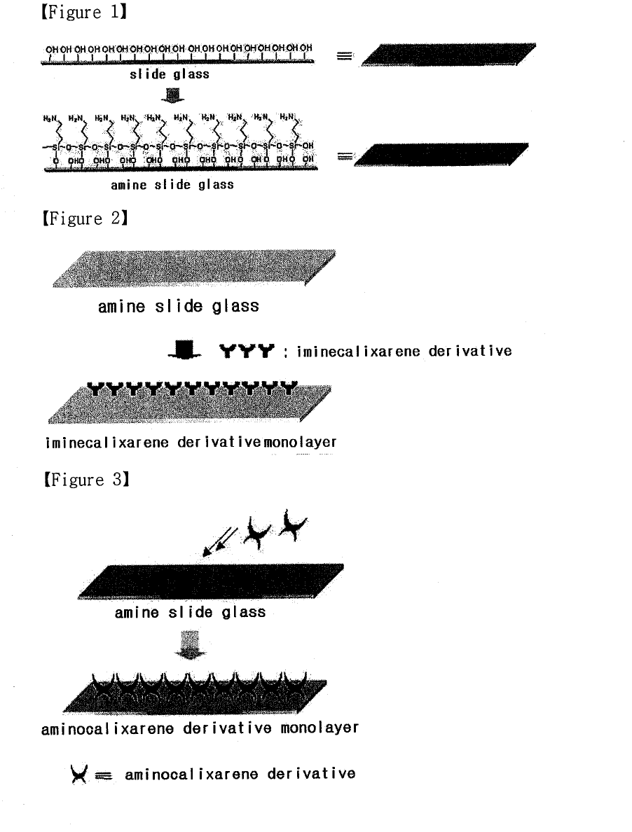

FIG. 1 shows a method of introducing amine functional groups onto a glass slide as one of the methods of introducing various functional groups onto a solid substrate in order to attach aminocalixarene derivatives to a solid substrate, and the specific preparation method is as follows:

The glass substrate wherein amine functional group is attached (glass slide glass) is prepared in the form of a glass substrate having an amine terminal group (amine slide glass or amine chip) through a chemical reaction on the surface of a glass substrate having sufficient silanol (—Si—OH) functional groups according to the method described in the paper (Langmuir, 1997, Vol. 13, pp 4305-4308; Langmuir, 1996, Vol. 12, pp 5338-5342).

FIG. 2 schematically shows the process of preparing a self-assembled iminecalixarene derivative monolayer of the present invention on a glass substrate.

The detailed method for preparing a self-assembled iminecalixarene derivative monolayer on a glass substrate wherein amine groups are attached is as follows:

First, the glass substrate wherein said amine functional group is attached (glass slide glass) is prepared in the form of a glass substrate having an amine terminal group (amine slide glass or amine chip) through a chemical reaction on the surface of a glass substrate having sufficient silanol (—Si—OH) functional groups according to the method described in the paper (Langmuir, 1997, Vol. 13, pp 4305-4308; Langmuir, 1996, Vol. 12, pp 5338-5342). The thus-prepared glass substrate wherein the amine functional group is attached and the compound of formula 2 are added to a mixed solution of chloroform and THF dissolved in a concentration of 0.1-5 mM (CHCl₃:THF=9:1). After 1˜5 hours, it is washed with chloroform, acetone and ethanol in that order and dried, and then an iminecalixarene self-assembled monolayer as FIG. 2 is completed. The formation of said monolayer is confirmed using infrared external reflection spectroscopy. As said glass substrate, all types of slide glasses or glasses being sold can be used. Since living substances such as protein are mostly prepared by being dissolved in a buffer solution wherein the pH is between 7˜8, it has been confirmed that there is no problem in using it immediately in the form of an imine bonding when being used for such purposes.

FIG. 3 shows a method of attaching a compound having an aldehyde functional group among the derivatives of the formula 6 or 7 to said amine slide glass, one of the solid substrates used in preparing a protein monolayer through protein fixation. The glass substrate wherein the amine functional group is attached, i.e. amine slide glass and the compound of formula 6 or 7 are added to a mixed solution of chloroform and THF dissolved in a concentration of 0.1-5 mM (CHCl₃:THF=9:1). After 1˜5 hours, it is washed with chloroform, acetone and ethanol in that order and dried, and then an aminocalixarene self-assembled monolayer as FIG. 3 is completed. The formation of said monolayer is confirmed using infrared external reflection spectroscopy. As said glass substrate, all types of slide glasses or glasses being sold can be used. Since living substances such as protein are mostly prepared by being dissolved in a buffer solution wherein the pH is between 7˜8, it has been confirmed that there is no problem in using it immediately in a state wherein the aminocalixarene derivative is combined with the amine slide glass through an imine bonding when being used for such purposes. For the purpose of developing products required to be used for a long period, an aminocalixarene derivative monolayer is used that is prepared by the well-known reduction reaction that reduces an imine to an amine functional group by putting the imine bond wherein a derivative is bound with a solid substrate for 1˜5 minutes in a THF or methanol solvent wherein BH₃/THF or 1-3% NaBH₄ is dissolved.

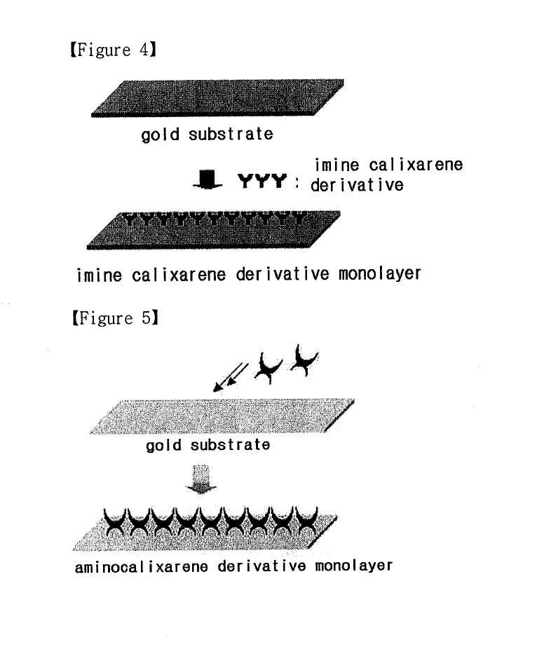

FIG. 4 schematically shows the process wherein a self-assembled iminecalixarene derivative monolayer is prepared on a gold substrate. The detailed method for preparing a self-assembled iminecalixarene derivative monolayer on a gold substrate is as follows:

A solution is prepared by dissolving a compound, wherein thiol is attached among the compounds of formula 2 or 4, in an organic solvent such as chloroform (CHCl3), etc. in a concentration of 0.1-5 mM. After a gold substrate is put into the thus-prepared solution and left for 1˜5 hours, it is taken out and washed with chloroform, acetone and ethanol in that order and dried, and then a self-assembled monolayer of iminecalixarene derivative as FIG. 2 is completed. Said gold substrate may be any type of gold thin film; however, in general, a substrate vacuum-plated with gold in a thickness of 50-200 nm after vacuum-plating glass, fused silica, silicon wafer, plastic substrate, etc. with chromium (Cr) or titanium (Ti), etc. in 2-10 nm, is used. It is preferable to put the thus-prepared gold substrate in a piranha solution (strong sulfuric acid:30% hydrogen peroxide=3:1) for about 1 minute immediately before use, and then wash it with purified water and dry it by blowing nitrogen. The formation of said monolayer is confirmed using infrared external reflection spectroscopy.

In addition, the present invention provides a method of fixing an unmodified protein by multiionic/multimolecular-recognizing the ammonium group of the protein or one or more of the guanidine groups in arginine, using four nitrogens of the iminecalixarene derivative.

In addition, the fixation method of the present invention is an original, fundamental technology for preparing all types of products using protein fixation such as diagnosis biochips, protein chips for research use, etc., and no other research results, not even similar ones, have been published in this regard all over the world until now.

FIG. 5 schematically shows the process that a self-assembled aminocalixarene derivative monolayer is prepared on a gold substrate. The detailed method for preparing a self-assembled aminocalixarene derivative monolayer on a gold substrate is as follows:

A solution is prepared by dissolving a compound, wherein thiol is attached among the compounds of formula 6 or 7, in an organic solvent such as chloroform (CHCl₃), etc. in a concentration of 0.1-5 mM. After a gold substrate is put into the thus-prepared solution and left for 1˜5 hours, it is taken out and washed with chloroform, acetone and ethanol in that order and dried, and then a self-assembled monolayer of aminocalixarene derivative as FIG. 5 is completed. Said gold substrate may be any type of gold thin film; however, in general, a substrate vacuum-plated with gold in a thickness of 50-200 nm after vacuum-plating glass, fused silica, silicon wafer, plastic substrate, etc. with chromium (Cr) or titanium (Ti), etc. in 2-10 nm, is used. It is preferable to put the thus-prepared gold substrate in a piranha solution (strong sulfuric acid:30% hydrogen peroxide=3:1) for about 1 minute immediately before use, and then wash it with purified water and dry it by blowing nitrogen. The formation of said monolayer is confirmed using infrared external reflection spectroscopy.

In addition, the present invention provides a method of fixing an unmodified protein by multiionic/multimolecular-recognizing the ammonium group of the protein or one or more of the guanidine groups in arginine.

In addition, the fixation method of the present invention is an original, fundamental technology for preparing all types of products that use protein fixation such as diagnosis biochips, protein chips for research use, and no other research results, not even similar ones, have been published in this regard all over the world until now.

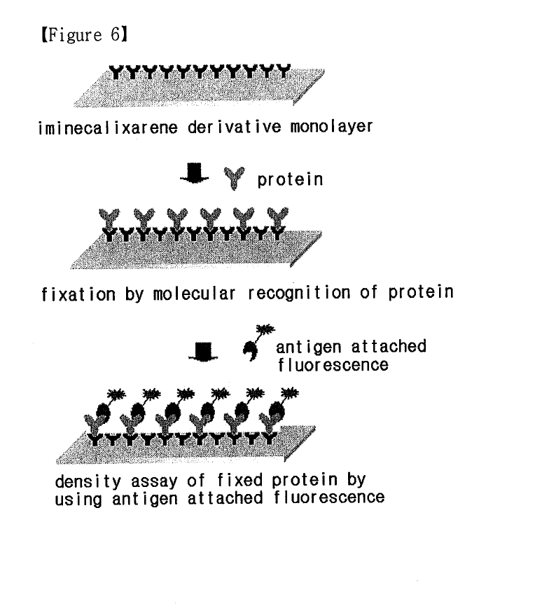

FIG. 6 is a diagram showing the process wherein after antibody proteins are fixed on the iminecalixarene derivative monolayer of the present invention by voluntary ionic/molecular recognition, Cy-5-antigen conjugate, which is the combination of the fixed antibody and Cy-5 fluorescence, is combined with them through antigen-antibody reaction. The density and activity of the fixed antibody proteins are confirmed by said process.

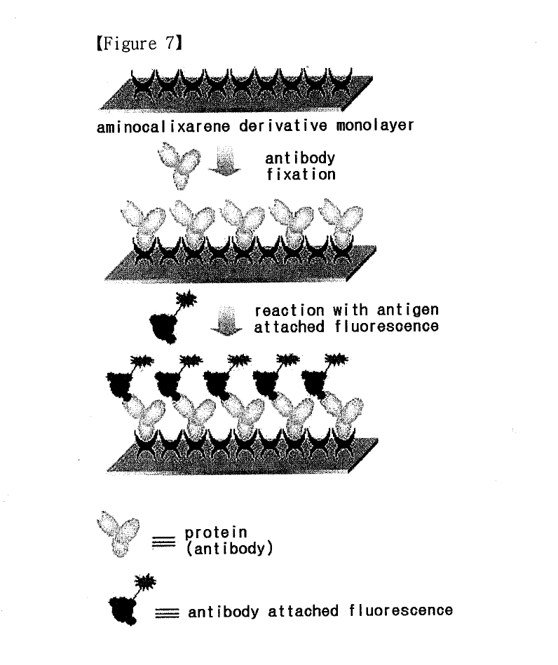

FIG. 7 is a diagram to confirm the density and activity of the fixed antibody proteins by showing the process wherein after antibody proteins are fixed on the aminocalixarene derivative monolayer of the present invention by voluntary ionic/molecular recognition, Cy-5-antigen conjugate, which is the combination of the fixed antibody and Cy-5 fluorescence, is combined with them through antigen-antibody reaction.

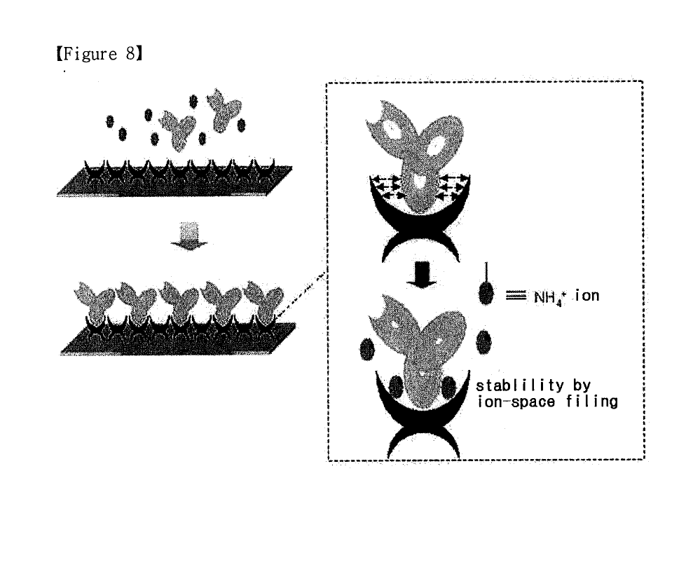

FIG. 8 is a diagram showing that during the protein fixation presented in the FIG. 7, when a terminal which can form a hydrogen bond such as alginine or an ammonium group of a protein, etc. is recognized by the aminocalixarene derivative, the protein fixation occurs with the remaining empty space filled with cations such as ammonium ions.

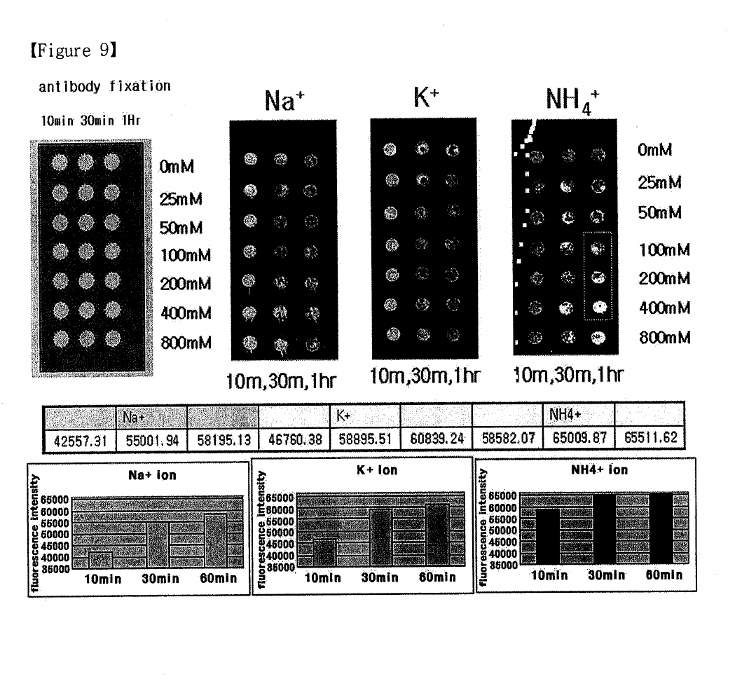

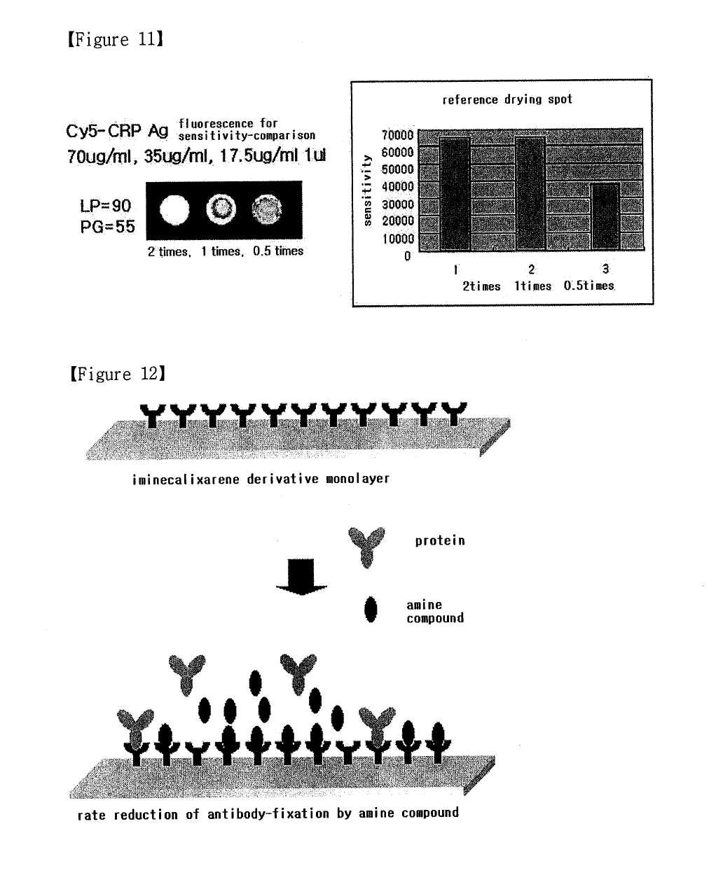

FIG. 9 shows the actual research result obtained by using the antigen (Ag) and antibody (Ab) of C-reactive protein (CRP) on an iminecalixarene monolayer, as shown in FIG. 6. Said result shows the speed and density at which proteins are fixed on the iminecalixarene derivative monolayer of the present invention, and at the same time the activity analysis of the fixed protein. For the protein fixation, three kinds of ions different in size a bit, Na⁺, K⁺, NH₄⁺, and antibody proteins of the same concentration (3 times the theoretically maximum fixation density (1.4 μg/cm²), 45 μg/me 3 uL) are used. Also, antibody proteins are fixed over a different fixation time of 10 minutes, 30 minutes, and 1 hour. After the fixed antibody protein is combined with enough fluorescent attached antigen proteins through antibody-antigen reaction, both activity and density of the fixed antibody are analyzed. The fluorescence analysis is confirmed using scanner (GSI lite, U.S.A.). According to the analysis result, it is confirmed that for both cases of using Na⁺ ion and using K⁺ ion in a concentration of 100 mM to 800 mM, as the fixation time of antibody proteins lengthens 10 minutes, 30 minutes, and 1 hour, fluorescence sensitivity gradually increases and the maximum fluorescence sensitivity is achieved at 1 hour. The color of fluorescence sensitivity nears white at the point as high as the maximum fluorescence sensitivity. In case of using NH₄⁺ ion, it is confirmed that just after 10 minutes of protein fixation, about 90% of the maximum fluorescence is achieved, showing that the fixation is about the same level as after 30 minutes to 1 hour. In addition, the same result is obtained in an ion concentration of 100 mM to 800 mM, which shows that in various ion concentration conditions, especially when using NH₄ ion, the protein fixation occurs in 10 to 30 minutes at a superhigh speed. In addition, the antibody protein used for the fixation is one to which no alteration was made. This result is the same as that of the theoretically maximum fluorescence sensitivity, shown in FIG. 11, measured after coating fluorescent attached antigens in the number that can form 1:1 bonding with the theoretically maximum fixation amount of antibody proteins (1.4 μg/cm²) and drying them, which shows that the theoretically maximum amount of proteins is fixed in just 10 to 30 minutes achieving excellent activity, which indicates that the iminecalixarene derivative monolayer for protein fixation developed according to the present invention fixes unmodified proteins as a monolayer in a minimum time at a superhigh speed. Particularly, while conventional protein chips cause much physical absorption in protein fixation, thus having problems with reproducibility and having difficulty in being put on the market, the present invention is a novel technology that achieves reproducibility in protein chip preparation by removing most of the physical absorption, which is supported by the fact that physical absorption hardly occurs in a high ion concentration of 400 to 500 mM or above.

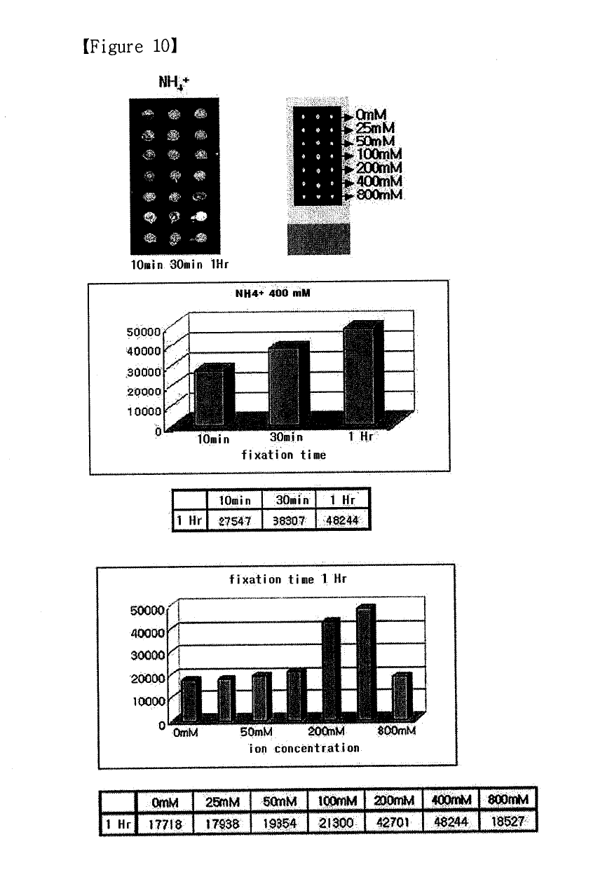

FIG. 10 shows the actual research result obtained by using the antigen (Ag) and antibody (Ab) of C-reactive protein (CRP) on an aminocalixarene monolayer, as shown in the FIG. 7. Said result shows the speed and density at which proteins are fixed on the aminocalixarene derivative monolayer of the present invention, and at the same time the activity analysis of the fixed protein. For the protein fixation, NH₄⁺ ion and antibody proteins of the same concentration (3 times the theoretically maximum fixation density (1.4 μg/cm²), 3 μl of 45 μg/ml proteins are coated each) are used. Also, antibody proteins are fixed over a different fixation time of 10 minutes, 30 minutes, and 1 hour. After the fixed antibody protein is combined with enough fluorescent attached antigen proteins through antibody-antigen reaction, both activity and density of the fixed antibody are analyzed. The fluorescence analysis is confirmed using scanner (GSI lite, U.S.A.).

According to the analysis result, it is confirmed that in case of fixation using NH₄⁺ ion in a concentration of 0-100 mM, about 40% of the maximum fluorescence is achieved after about 1 hour of protein fixation. On the other hand, in case of fixation using NH₄⁺ ion in a concentration of 400 mM, the fluorescence is almost the same in 1 hour as the theoretically maximum fluorescence shown in the FIG. 11. Especially in a concentration of 400 mM, about 65% of the maximum fluorescence is achieved just after about 30 minutes of protein fixation, which is the actual experiment result showing that in a short time of 30 minutes to 1 hour protein fixation is performed at a high density to a level of a protein monolayer. Also, said results show that the higher the density of ions filling the space remaining after functional groups of the protein are recognized is, the faster the fixation proceeds, which is the actual research result confirming the effect of ion concentration presented in the diagram of FIG. 8. However, regarding the result for a concentration of 800 mM, which is lower than that of 400 mM, it is widely known that in a high ion concentration, protein often loses activity with alteration being made to the active site of the protein. The result for 800 mM presented in the FIG. 10 shows that the fixation occurred at a high speed and density, but the activity of the fixed protein considerably decreased. For the present experiment the antibody protein used was one without having had any treatment.

In addition, the present invention relates to a protein chip prepared by forming a protein monolayer (i.e., protein chip) on an aminocalixarene derivative monolayer at superhigh speed, high density, and high reproducibility using an unmodified protein, and the preparation method thereof.

FIG. 11 shows the result of an actual experiment wherein after coating fluorescent attached antigens of the same amount as that of the antigen (35 ng/0.0314 cm², dry spot having a diameter of 2 mm) making 1:1 combination with the antibody fixed on a solid substrate at the maximum density (1.4 μg/cm²) and drying them, the fluorescence is measured, and also, after coating two times and half time more of said fluorescent attached antigens, the fluorescence is measured. When the same amount is coated, the brightness of the fluorescent attached antigen is the same as that of the antigen combining with the antibody fixed in the presence of NH₄⁺ of 100 mM or more for 30 minutes in the FIG. 9, and it is shown that in case of using half of the amount, the fluorescence has decreased to half as compared with when the same amount is used.

FIG. 12 is a diagram of a competitive reaction showing the fixation of the antibody protein occurs actually by ion recognition. It shows that at PH 7, amine compounds exist 100% in the form of ammonium ion and if such amine compounds are added in a suitable amount, the protein fixation can be inhibited.

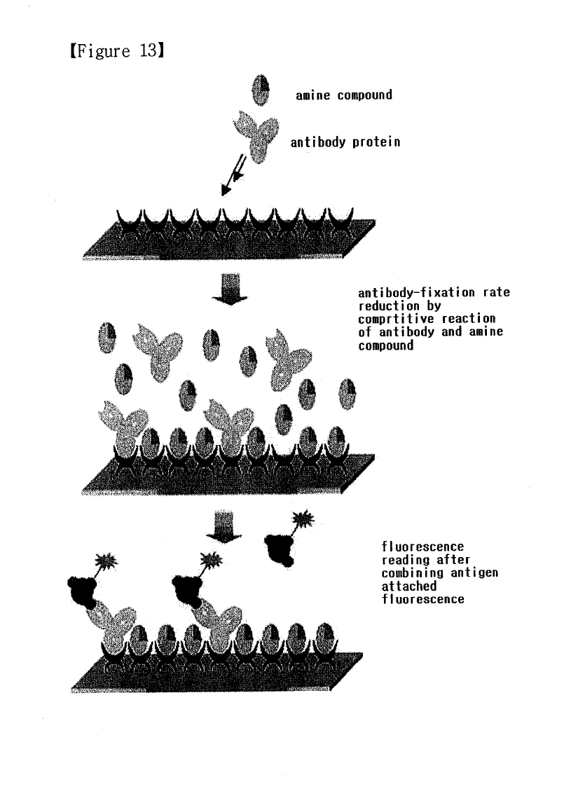

FIG. 13 is a diagram of a competitive reaction showing a process wherein compounds having an ammonium ion are added at the fixation step and its effect on the fixation density is analyzed in order to confirm whether antibody protein fixation occurs actually by ionic recognition. It shows that at PH 7, amine compounds exist 100% in the form of ammonium ion and if such amine compounds are added in a suitable amount, the protein fixation can be inhibited.

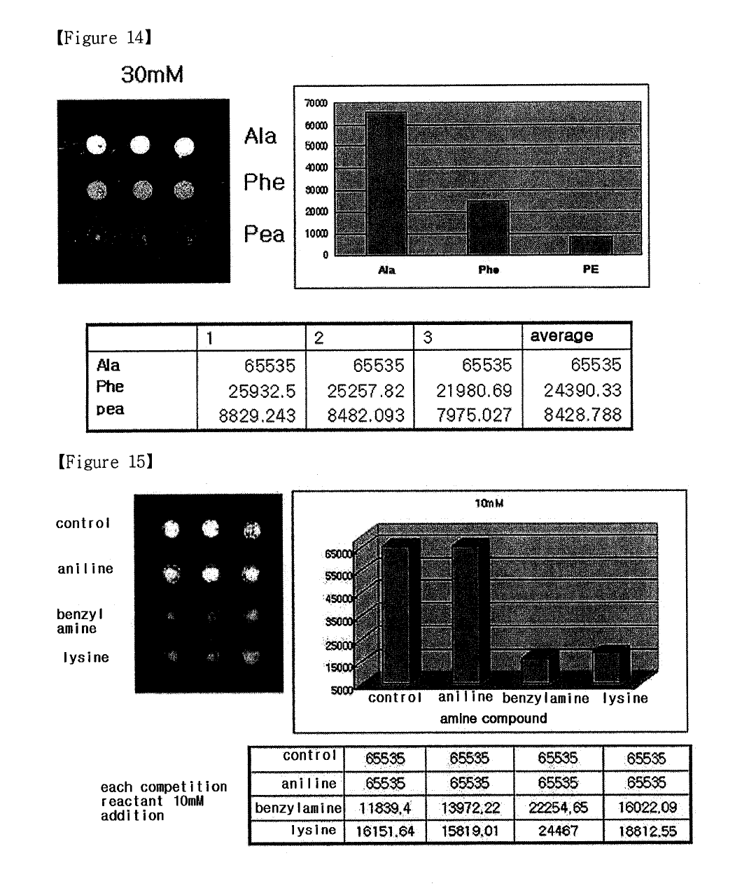

FIG. 14 shows the result of a research actually carried out according to the diagram of FIG. 12. Using phenetylamine, which is one of the agents fastest recognized by the iminecalixarene derivative monolayer, phenylalanine, one of the amino acids, which ranks the next, and alanine, which is recognized relatively slowly, the same experiment as that of the FIG. 9 was carried out after simply mixing each amine compound to a concentration of 30 mM, and then the fixation result were compared. When phenethylamine and phenylalanine, which are easily recognized, are used, the fixation proceeds at a level of 1/10 to ⅓ as compared to the fixation without using them, whereas in case of alanine (measurement result by NMR), which is recognized slowly, the fixation speeds are not much different.

Such results show that the protein fixation proceeds actually by ion recognition, and especially if there is a p-p stacking interaction between the iminecalixarene ring and the benzene ring of amine compounds, amine compounds are more easily recognized by the iminecalixarene derivative, and thus inhibit the protein fixation faster, which shows that on the iminecalixarene derivative monolayer of the present invention, proteins are mostly fixed by the recognition of amine groups at the outside of the protein.

FIG. 15 shows the result of a research actually carried out according to the FIG. 13. diagram. It is the actual research result of an antibody protein fixation reaction carried out by adding to the aminocalxiarene derivative monolayer, 10 mM of benzylamine having an ammonium functional group, lysine having an ammonium terminal, and aniline having an amine functional group which does not change to an ammonium ion in a buffer solution of around PH 7. For comparison with the result of the competitive reaction, the fixation reaction without adding any amine compounds was also carried out. (control). After combining the fluorescent attached antigen protein with the fixed antibody protein with the ratio of 1:1, the density of the fixed antibody protein was analyzed. In case of adding 10 mM of lysine and benzylamine, the antibody protein fixation proceeded only about ¼, compared to the control, showing that the protein fixation was seriously inhibited by the compounds having an ammonium functional group. In case of adding arginine having a guanidine terminal, the fixation was also inhibited. On the other hand, in case of adding aniline, which does not change to ammonium under fixation conditions, the fixation density is almost the same as that of the control, showing that aniline never inhibits the fixation process. Such results are actual experiment results showing that in antibody protein fixation, ammonium functional groups, etc. outside the protein are actually ionic/molecular-recognized by the aminocalixarene derivative.

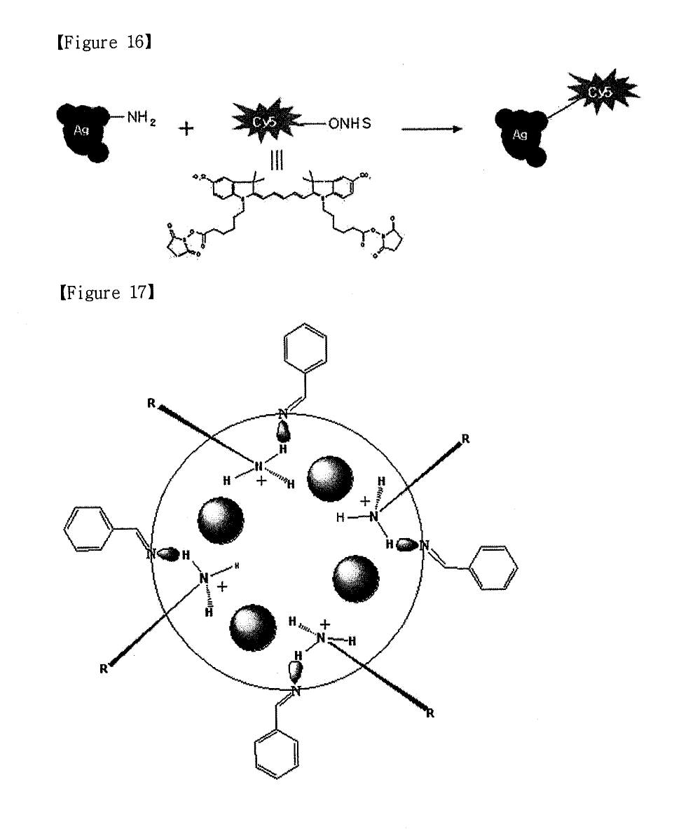

FIG. 16 is a diagram that shows attaching Cy-5 fluorescence (Telechem, U.S.A.) to an antigen protein that combines with an antibody protein for fluorescence reading.

FIG. 17 is a diagram showing the state wherein the iminecalixarene derivative of the present invention has recognized four amine compounds (ammonium ions in a solution phase). Spheres existing between ammoniums are anions. It was confirmed that four amine compounds are recognized by molecular recognizing various amine compounds irreversibly.

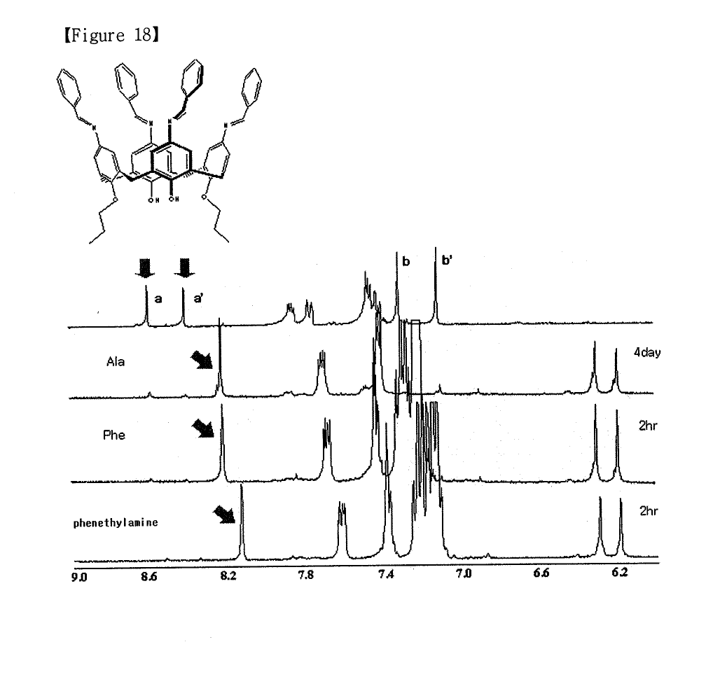

FIG. 18 is the NMR result showing that four amine compounds in the form of ammoniums were actually recognized by the iminecalixarene derivatives in a solution phase. The NMR result shows that four imine hydrogens in the iminecalixarene derivative moved to the same position and eight hydrogens in the calixarene also moved about 1 ppm, which shows that the ammonium ion recognition is irreversible not in a reversible equilibrium. In addition, the result shows that phenylalanine or phenethylamine, which is an amine compound having a benzene ring, are recognized tens of times faster than those without a benzene ring, supporting the competitive reaction result of FIG. 14.

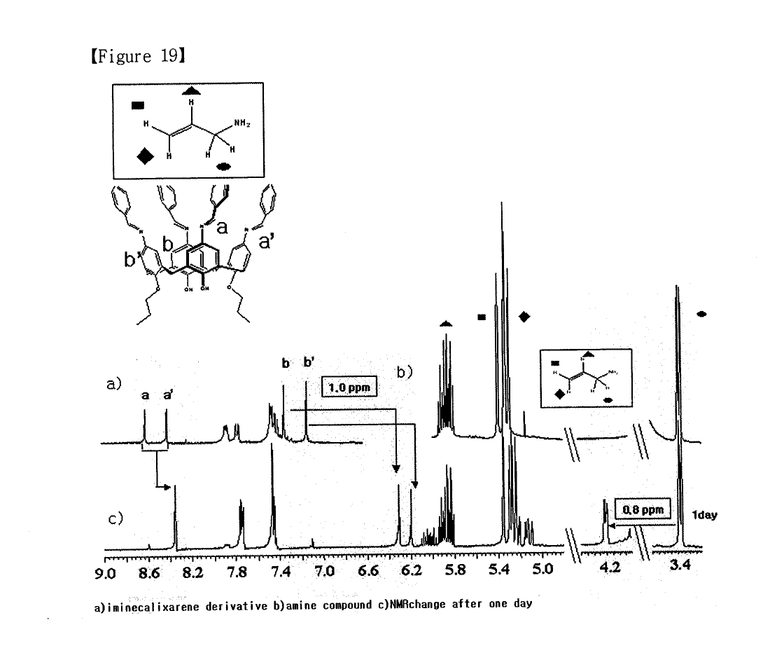

FIG. 19 is another experiment result showing that when using an allylamine, after four allylamines are recognized, eight(4×2=8) hydrogens of CH₂ of the allylamine moved 0.8 ppm and hydrogens attached to double bonds moved to another position at the rate of four allylamines, thus showing that four amine compounds are irreversibly recognized. The imine hydrogen in the iminecalixarene and the hydrogen in the calixarene have a moving pattern similar to that of the case when other amine compounds of the FIG. 18 are recognized.

From said results, it can be found that in the present invention four amine compounds, i.e. ammonium functional groups are irreversibly recognized on the surface of an iminecalixarene derivative monolayer and that the protein is fixed at a high density with ammonium functional groups outside the protein being recognized irreversibly on the surface of an iminecalixarene derivative monolayer.

In addition, the present invention provides a superhigh speed fixation technology, novel iminecalixarene derivatives or aminocalixarene derivatives that make possible a fixation of proteins that have undergone no treatment, i.e. unmodified proteins, and monolayers thereof, and the protein chip using said monolayers.

Hereinafter, the present invention is described in more detail by the following examples. However, the present invention is not restricted thereto.

### MODE FOR INVENTION

### EXAMPLE 1

### Synthesis of 5,11,17,23-tetraaminocalix[4]arene

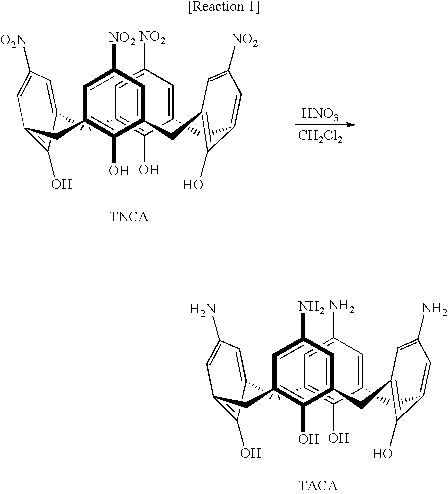

A light-yellow solid product, 5,11,17,23-tetraaminocalix[4]arene (TACA) is obtained in the yield of 75% by having 5,11,17,23-tetranitrocalix[4]arene (TNCA) as a starting material and synthesizing it according to the synthesizing method presented in the following cited reference.

[Cited Reference: Van Wagenigen, A. M. A.; Snip, E.; Verboom. W.; Reinhoudt, D. N.; Boerrigter, H.; Liebigs Ann/Recueil 1997. pp 2235-2245].

### EXAMPLE 2

### Synthesis of 5,11,17,23-tetra-2,4-dimethoxybenzyliminecalix[4]arene

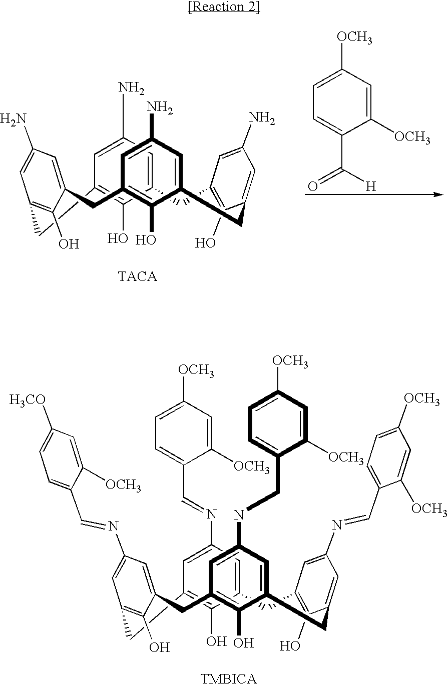

Experiment is prepared by putting TACA (100 mg, 0.2 mmol) and magnetic bar in a dried round-bottom flask. After adding 15 ml of acetonitrile in the reaction vessel is added, it is mixed. After 2,4-dimethoxybenzaldehyde (330 mg, 2.4 mmol) is put, it is mixed for two hours under nitrogen exchange at room temperature. After the reaction, the solvent is removed under reduced pressure by decompressed filtering; the reaction product is dissolved in 3 ml of CH₂Cl₂; and then 15 ml of n-hexane is added to obtain a light-brown solid product. The product is dissolved in 3 ml of CH₂Cl₂ once again, and 15 ml of n-hexane is slowly added to obtain 196 mg (yield 88%) of a light-brown solid product, TMBICA (5,11,17,23-tetra-2,4-dimethoxybenzyliminecalix[4]arene).

¹H NMR (300 MHz, CDCl₃): δ 10.0(s, 4H, OH), 8.66(s, 4H N═CH), 7.96(d, 4H,ArH) 6.98(s, 8H, ArH), 6.56˜6.37(m, 8H, ArH) 4.3(d, 4H, ArCH2Ar J=13 Hz), 3.81(s, 12H, OCH₃), 3.73(s, 12H, OCH₃), 3.58(d, 4H, ArCH₂Ar J=13 Hz)

### EXAMPLE 3

### Synthesis of 5,11,17,23-tetra-2,4-dimethoxybenzyliminecalix[4]arene-1,3-dihexanal

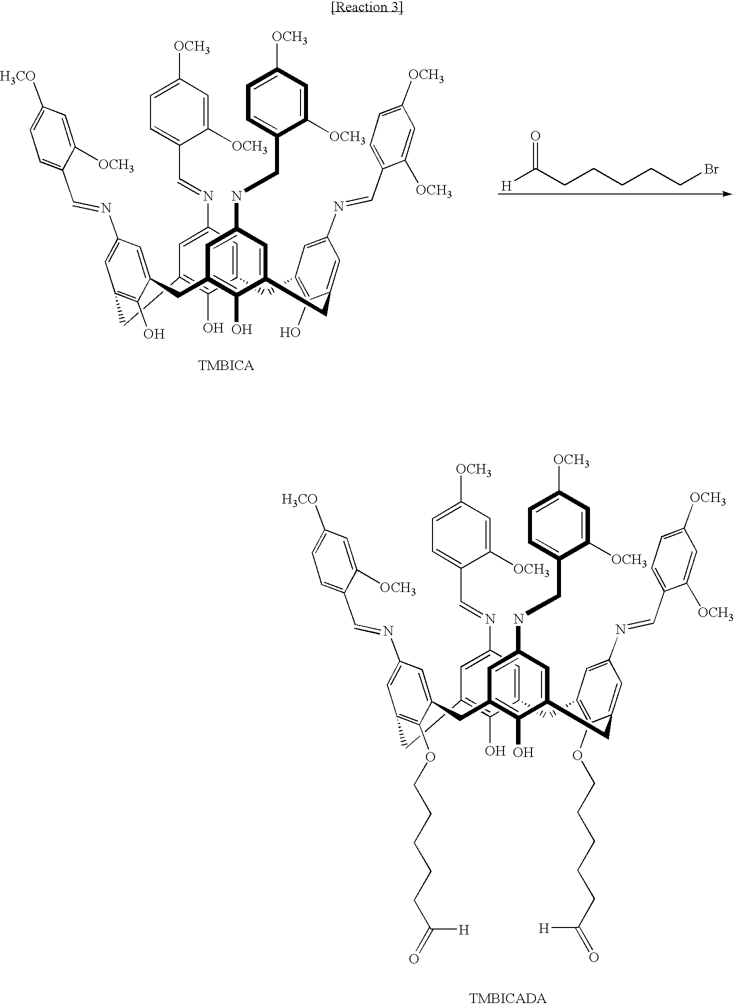

After a magnetic bar, TMBICA (100 mg, 0.1 mmol), K₂CO₃ (145 mg, 1.1 mmol), and sodium iodide (142 mg, 0.9 mmol) are put in that order in a dried round-bottom flask, it is dried under decompressed pressure, and 15 mg of anhydrous acetonitrile is added to the reaction vessel under nitrogen exchange, and then it is mixed on a heater. 6-bromohexanal (113 mg, 0.6 mmol) is added, and the temperature of the reaction vessel is raised to 80° C. Then, it is mixed for 24 hours. After the reaction, the reaction product is cooled down to room temperature, and the solvent is removed under decompressed pressure. To remove the additional insoluble solid product obtained during the reaction, the reaction product is dissolved in 10 ml of CH₂Cl₂, and the insoluble solid product is removed by decompressed filtering. The filtered solution is decompressed to remove the solvent.

After the reaction product is dissolved in 2 ml of CH₂Cl₂, 15 ml of n-hexane is slowly added, and then the reaction product is extracted. The extracted product is decompressed and filtered to obtain a yellow solid product. After this product is dried, it is dissolved in 2 ml of CH₂Cl₂ for refining. Then, the reaction product is slowly extracted by slowly adding 20 ml of n-hexane slowly to obtain 100 mg (yield 85%) of a light-yellow solid product, TMBICADA (5,11,17,23-tetra-2,4-dimethoxybenzyliminecalix[4]arene-1,3-dihexanal).

¹H NMR (300 MHz CDCl₃): δ 9.76(s, 2H, CHO), 8.79(s, 2H, n=CH) 8.58(s, 2H, N═CH) 8.04(d, 2H, ArH) 7.88(d, 2H, ArH), 7.25(s, 4H, ArH), 6.84(s, 4H, ArH) 6.55˜6.35(m, 8H, ArH), 4.32(d, 4H, ArCH₂Ar, J=13 HZ), 4.02(t, 4H, OCH₂), 3.90˜3.17(m, 24H, OCH₃), 3.46(d, 4H ArCH₂Ar J=13 Hz), 2.56(t, 4H, CH₂CHO) 2.16˜1.6(m 12H, CH₂CH₂)

### EXAMPLE 4

### Synthesis of 5,11,17,23-tetra-2,4-methoxybenzyliminecalix[4]arene-1,3-butylbromide

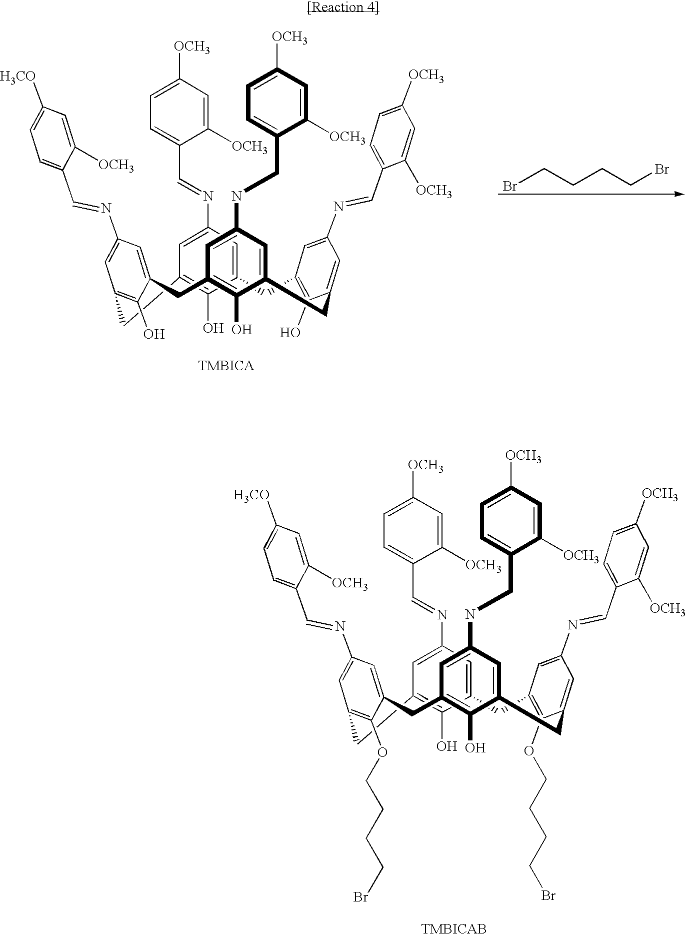

After TMBICA (500 mg, 0.46 mmol), anhydrous K₂CO₃ (636 mg, 4.6 mmol) and sodium iodide (620 mg, 4.14 mmol) are put in a dried round-bottom flask and then 160 ml of anhydrous acetonitrile is added, it is mixed for 15 minutes at room temperature. 1,4-dibromobutane (1.0 g, 0.55 ml, 4.6 mmol) is added to the reaction vessel and the temperature of the reaction vessel is raised to 80° C. Then, it is reacted for 24 hours. After the reaction vessel is cooled down to room temperature, the solvent is decompressed and removed, and the remaining product is dissolved with 150 ml of CH₂Cl₂. Solid precipitates obtained during the reaction, such as KBr, KI, K₂CO₃, are removed by being decompressed and filtered and the filtered solution is decompressed to remove the solvent. After being extracted with ethyl acetate/n-hexane, it is decompressed and filtered to obtain a light-brown solid product, 5,11,17,23-tetra-2,4-methoxybenzyliminecalix[4]arene-1,3-butylbromine (TMBICAB, 5,11,17,23-tetra-2,4-methoxybenzyliminecalix[4]arene-1,3-butylbromide). This solid is recrystallized with CHCl₃/n-hexane to obtain a light-yellow TMBCIAB (490 mg, 79%).

¹H NMR (300 MHz CDCl₃): δ 8.79(s, 2H, N═CH), 8.55(s, 2H, N═CH), 8.06(d, 2H, ArH) 7.90(d, 2H, ArH), 7.26(s, 4H, ArH), 6.85(s, 4H, ArH) 6.61˜6.46(m, 8H, ArH), 4.33(d, 4H, ArCH₂Ar, J=13 HZ), 4.03(t, 4H, OCH₂), 3.94˜3.21(m, 24H, OCH₃), 3.45(d, 4H ArCH₃Ar J=13 Hz), 3.32(t, 4H, CH₂Br) 2.41˜2.34(m, 4H, OCH₂CH₂), 2.24-2.18(m, 4H, CH₂CH₂Br)

### EXAMPLE 5

### Synthesis of 5,11,17,23-tetra-2,4-methoxybenzyliminecalix[4]arene-1,3-bytylmercaptane

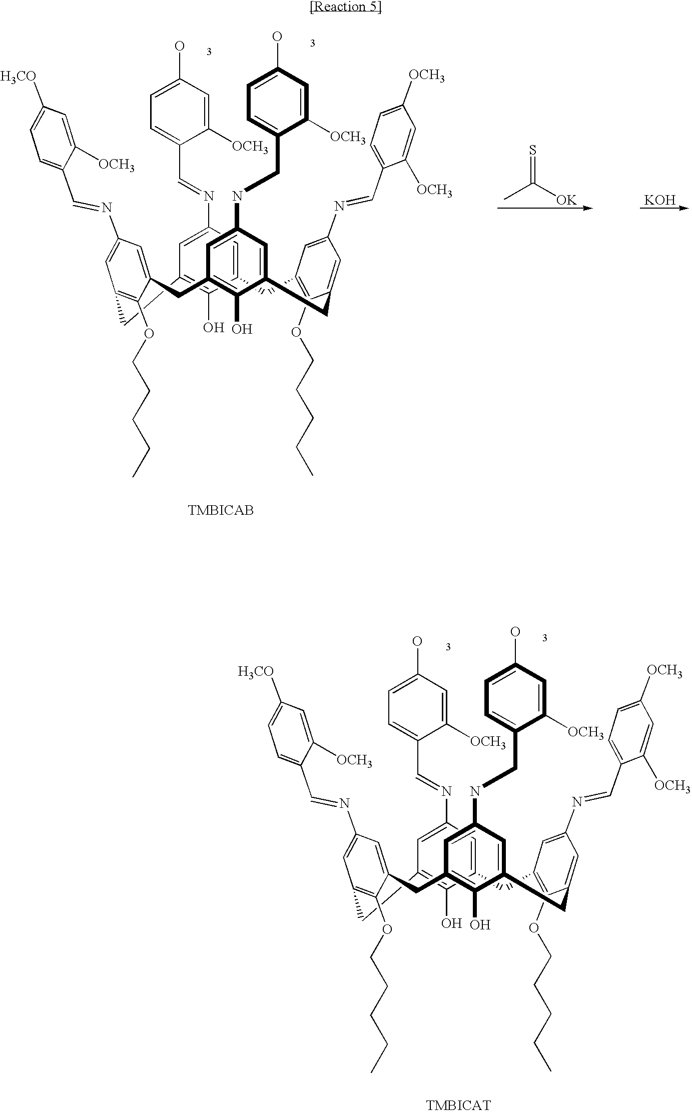

After TMBICAB (500 mg, 0.37 mmol) and potassium thioacetate (170 mg, 1.48 mmol) are put in a dried round-bottom flask, and dissolved in 50 ml of acetone, it is sonic reacted for 90 minutes at room temperature under nitrogen exchange. After the reaction, the solvent is removed under decompressed pressure, and the remaining product is dissolved with 30 ml of CH₂Cl₂. Then, the precipitate that is not dissolved is decompressed and filtered, and then the filtered solution is cleaned with water twice and the organic layer is separated and dried with MgSO₄. The solid obtained by depressing and filtering the organic layer and decompressing and drying the filtered solution is recrystallized using ethyl acetate/n-hexane, and decompressed and filtered to obtain a light-brown solid crystal. The thus-obtained solid crystal is put in the round-bottom flask, and dissolved in a mixed solution in a ratio of CH₂Cl₂:methanol=5:1, and sonic reacted at room temperature under nitrogen exchange. After 1 minute, 1.0 M KOH (1.5 ml, 1.5 mmol) is added, and it is sonic reacted for 30 minutes. After the reaction, the solvent is removed under decompressed pressure, and then it is dissolved in 4 ml of CH₂Cl₂ and washed with 0.1 M HCl solution once. After the organic layer is separated and dried with MgSO₄, it is decompressed and filtered. The solvent of the filtered solution is removed under decompressed pressure and 25 ml of n-hexane is slowly added to obtain a light-yellow 5,11,17,23-tetra-2,4-methoxybenzyliminecalix[4]arene-1,3-bytylmercaptane (TMBICAT). The thus-obtained TMBICAT is recrystallized with ethyl acetate/n-hexane to obtain a white solid crystal, TMBICAT (370 mg, yield 79%).

¹H NMR (300 MHz CDCl₃): δ 3.78(s, 2H, N═CH), 8.54(s, 2H, N═CH), 8.03(d, 2H, ArH) 7.87(d, 2H, ArH), 7.24(m, 4H, ArH), 6.83(s, 4H, ArH) 6.59˜6.43(m, 8H, ArH), 4.33(d, 4H, ArCH₂Ar, J=13 HZ), 4.03(t, 4H, OCH₂), 3.93˜3.21(m, 24H, OCH₃), 3.45(d, 4H ArCH₂Ar J=13 Hz), 3.04(t, 4H, CH₂SH) 2.39(m, 4H, OCH₂CH₂), 2.21-2.14(m, 4H, CH₂CH₂)

### EXAMPLE 6

### Synthesis of 5,11,17,23-tetra-2,5-dimethoxybenzyliminecalix[4]arene

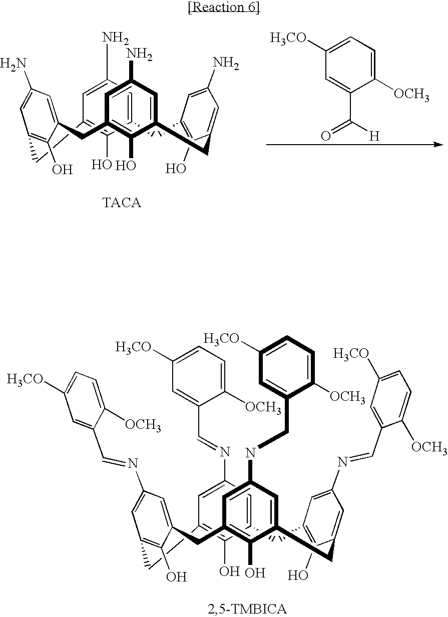

TACA (500 mg, 1.0 mmol) and magnetic bar are put in a dried round-bottom flask. After 60 ml of acetonitrile is added to the reaction vessel, it is mixed. After 2,5-dimethoxybenzaldehyde (1.65 g, 12 mmol) is put, it is mixed for two hours under nitrogen exchange at room temperature. After the reaction, the solvent is removed under reduced pressure by decompressed filtering; the reaction product is dissolved in 5 ml of CH₂Cl₂; and then 30 ml of n-hexane is added to obtain a light-brown solid product. The product is dissolved in 5 ml of CH₂Cl₂ once again, and 30 ml of n-hexane is slowly added to obtain 920 mg (yield 85%) of a light-brown solid product, 5,11,17,23-tetra(2,5-dimethoxy)benzyliminecalix[4]arene (2,5-TMBICA, 5,11,17,23-tetra-2,5-dimethoxybenzyliminecalix[4]arene).

¹H NMR (300 MHz, CDCl₃): δ 10.5(s, 4H, OH), 8.75(s, 4H N═CH), 7.57(d, 4H, ArH) 7.02(s, 8H, ArH), 6.86(m, 8H, ArH) 4.28(d, 4H, ArCH₂Ar J=13 Hz), 3.79(s, 12H, OCH₃), 3.72(s, 12H, OCH₃) 3.58(d,4H,ArCH₂Ar J=13 Hz)

### EXAMPLE 7

### Synthesis of 5,11,17,23-tetra-2,5-dimethoxybenzyliminecalix[4]arene-1,3-dihexanal

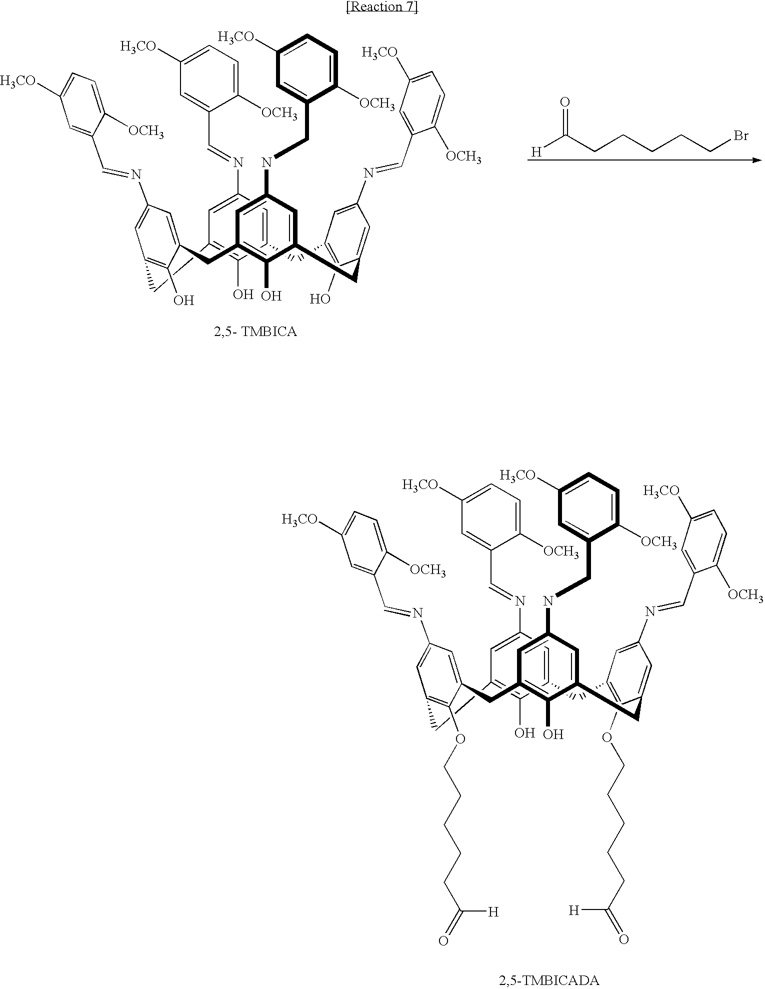

After a magnetic bar, 2,5-TMBICA (500 mg, 0.46 mmol), K₂CO₃ (625 mg, 4.6 mmol), and sodium iodide (630 mg, 4.2 mmol) are put in that order in a dried round-bottom flask, it is dried under decompressed pressure, and 130 ml of anhydrous acetonitrile is added to the reaction vessel under nitrogen exchange, and then it is mixed on a heater. 6-bromohexanal (520 mg, 2.76 mmol) is added, and the temperature of the reaction vessel is raised to 80° C. Then, it is mixed for 24 hours. After the reaction, the reaction vessel is cooled down to room temperature, and the solvent is removed under decompressed pressure. To remove the additional insoluble solid product obtained during the reaction, the reaction product is dissolved in 120 ml of CH₂Cl₂, and the insoluble solid product is removed by decompressed filtering. The filtered solution is decompressed to remove the solvent. After the reaction product is dissolved in 5 ml of CH₂Cl₂, 30 ml of n-hexane is slowly added to extract the reaction product. The extracted product is decompressed and filtered to obtain a yellow solid product. After this product is dried, it is dissolved in 5 ml of CH₂Cl₂ for refining. Then, the reaction product is slowly extracted by slowly adding 30 ml of n-hexane to obtain 510 mg (yield 87%) of a light-yellow solid product, 5,11,17,23-tetra-2,5-dimethoxybenzyliminecalix[4]arene-1,3-dihexanal (2,5-TMBICADA).

1H NMR (300 MHz CDCl₃): δ 9.75(s,2H,CHO), 8.86(s, 2H, N═CH) 8.66(s, 2H, N═CH) 7.55(d, 2H, ArH), 7.45(d, 2H, ArH), 7.09(s, 4H, ArH), 6.95(s, 4H, ArH), 6.90˜6.72(m, 8H, ArH), 4.32(d, 4H, ArCH2Ar, J=13 Hz), 4.03(t, 4H, OCH₂), 3.87˜3.65(m, 24H, OCH3), 3.45(d, 4H ArCH2Ar J=13 Hz), 2.55(t, 4H, CH2CHO), 2.10˜1.5(m 12H, CH2CH2)

### EXAMPLE 8

### Synthesis of 5,11,17,23-tetra-2,5-dimethoxybenzyliminecalix[4]arene-1,3-butylbromide

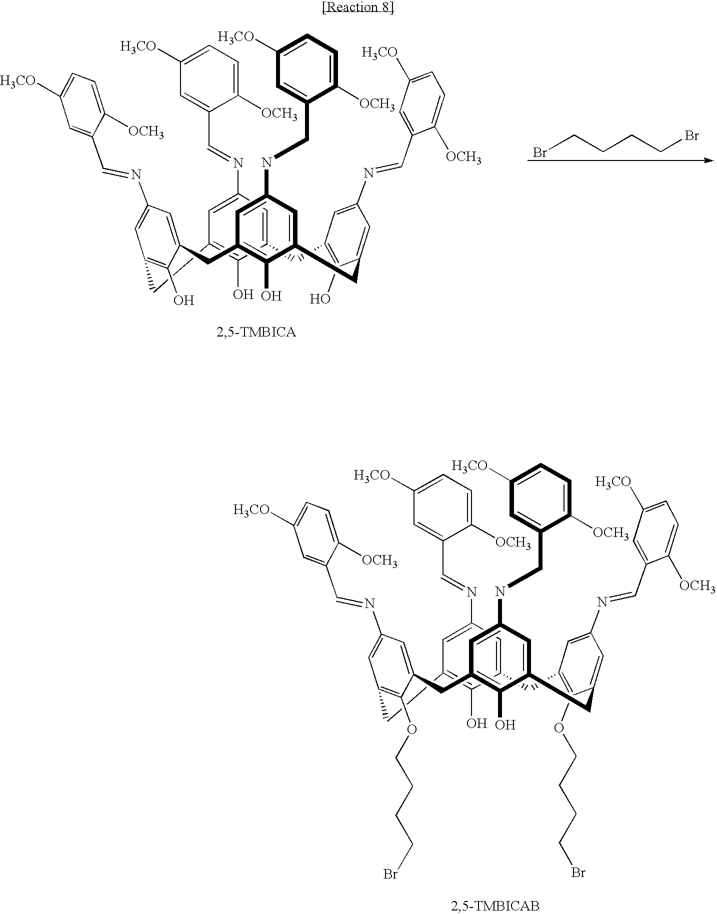

After 2,5-TMBICA (300 mg, 0.28 mmol), anhydrous K₂CO₃ (387 mg, 2.8 mmol) and sodium iodide (378 mg, 2.52 mmol) are put in a dried round-bottom flask, and 150 ml of anhydrous acetonitrile is added, it is mixed for 10 minutes at room temperature. 1,4-dibromobutane (600 mg, 0.34 ml, 2.8 mmol) is added to the reaction vessel and the temperature of the reaction vessel is raised to 80° C. Then, it is reacted for 24 hours. After the reaction, the reaction vessel is cooled down to room temperature; the solvent is decompressed and removed; and the remaining product is dissolved with 140 ml of CH₂Cl₂. Solid precipitates obtained during the reaction, such as KBr, KI, K₂CO₃, are removed by being decompressed and filtered and the filtered solution is decompressed to remove the solvent. After being extracted with ethyl acetate/n-hexane, it is decompressed and filtered to obtain a light-brown solid product, 5,11,17,23-tetra-2,5-dimethoxybenzyliminecalix[4]arene (2,5-TMBICA, 5,11,17,23-tetra-2,5-dimethoxybenzyliminecalix[4]arene-1,3-butylbromide). This solid is recrystallized with CHCl₃/n-hexane to obtain a light-yellow 2,5-TMBICA (300 mg, 79%).

¹H NMR (300 MHz, CDCl₃): δ 8.87(s, 2H, N═CH) 8.67(s, 2H, N═CH) 7.65(d, 2H, ArH), 7.49(d, 2H, ArH), 7.09(s, 4H, ArH), 6.95(s, 4H, ArH), 6.89˜6.71(m, 8H, ArH), 4.32(d, 4H, ArCH2Ar, J=13 Hz), 4.03(t, 4H, OCH₂), 3.87˜3.65(m, 24H, OCH3), 3.45(d, 4H ArCH2Ar J=13 Hz), 3.42.(t, 4H, CH₂Br), 2.41˜2.33(m, 4H, OCH₂CH₂), 2.23-2.17(m, 4H, CH₂CH₂Br)

### EXAMPLE 9

### Synthesis of 5,11,17,23-tetra-2,5-dimethoxybenzyliminecalix[4]arene-1,3-bytylmercaptane

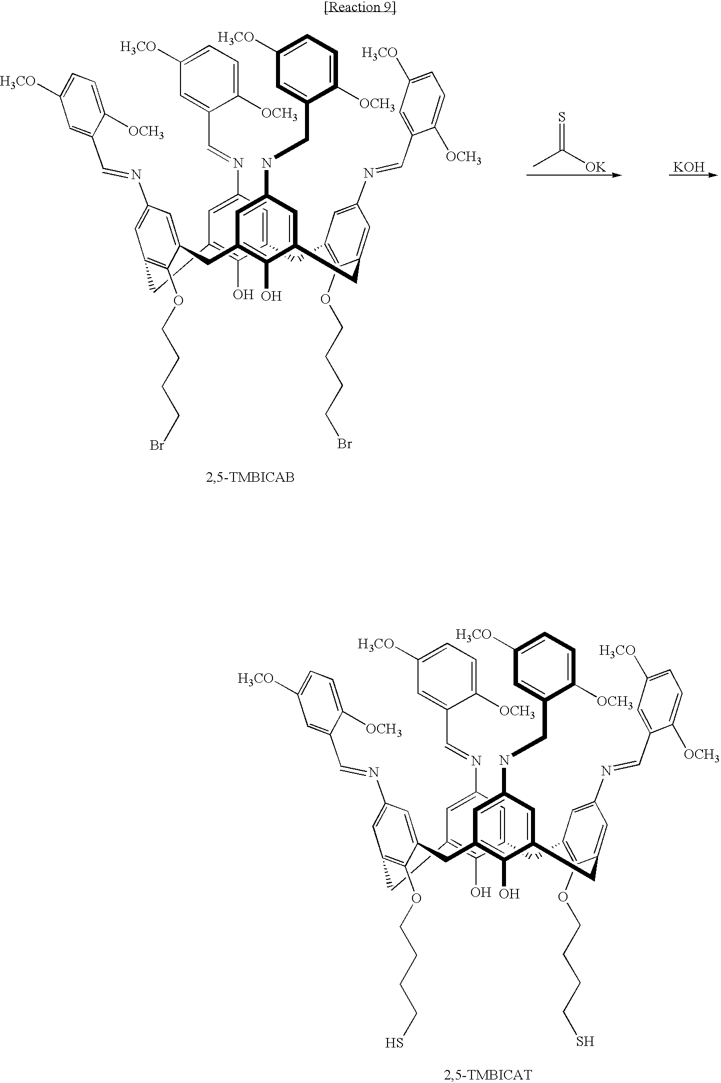

After 2,5-TMBICAB (500 mg, 0.37 mmol) and potassium thioacetate (200 mg, 1.75 mmol) are put in a dried round-bottom flask, and dissolved in 60 ml of anhydrous acetone, it is sonic reacted for 90 minutes at room temperature under argon exchange. After the reaction, the solvent is removed under decompressed pressure, and the remaining product is dissolved with 30 ml of CH₂Cl₂. Then, the precipitate that is not dissolved is decompressed and filtered, and then the filtered solution is cleaned with water twice and the organic layer is separated and dried with MgSO₄. Thereafter, the solid obtained by decompressing and filtering the organic layer and removing the filtered solution under decompressed pressure, and then drying the remaining product is recrystallized using ethyl acetate/n-hexane, and decompressed and filtered to obtain a light-brown solid crystal. The thus-obtained solid crystal is put in the round-bottom flask, and dissolved in a mixed solution in a ratio of CH₂Cl₂:methanol=5:1, and sonic reacted at room temperature under argon exchange. After 1 minute, 1.0 M KOH (1.5 ml, 1.5 mmol) is added, and it is sonic reacted for 30 minutes. After the reaction, the solvent is removed under decompressed pressure, and then it is dissolved in 5 ml of CH₂Cl₂ and washed once with 0.1 M HCl solution and water each. After the organic layer is separated and dred with MgSO₄, it is decompressed and filtered, and then the solvent of the filtered solution is removed under decompressed pressure. By slowly adding 30 ml of n-hexane, a solid product is extracted to obtain a light-yellow 5,11,17,23-tetra-2,5-methoxybenzyliminecalix[4]arene-1,3-bytylmercaptane (2,5-TMBICAT). The thus-obtained 2,5-TMBICAT is recrystallized with ethyl acetate/n-hexane to obtain a white solid crystal, 2,5-TMBICAT (385 mg, yield 82%).

¹H NMR (300 MHz CDCl₃): δ 8.88(s, 2H, N═CH) 8.67(s, 2H, N═CH) 7.67(d, 2H, ArH), 7.51(d, 2H, ArH), 7.10(s, 4H, ArH), 6.96(s, 4H, ArH), 6.89˜6.71(m, 8H, ArH), 4.33(d, 4H, ArCH2Ar, J=13 Hz), 4.03(t, 4H, OCH₂), 3.87˜3.65(m, 24H, OCH3), 3.46(d, 4H ArCH2Ar J=13 Hz), 3.09.(t, 4H, CH₂SH), 2.38˜2.34(m, 4H, OCH₂CH₂), 2.19-2.12(m, 4H, CH₂CH₂SH)

### EXAMPLE 10

Preparation of Iminecalixarene Derivative Monolayer as Shown in FIG. 2

A solution, wherein TMBICADA synthesized in example 3 is dissolved in an organic solvent such as CHCl₃, etc. in a concentration of 0.1˜5 mM, is prepared. As shown in FIG. 2, a slide glass (amine chip) wherein an amine functional group is attached is put in the prepared solution for 1˜24 hours, and then taken out and washed with chloroform, acetone and water each, and dried to prepare the iminecalixarene monolayer shown in FIG. 2. Other iminecalixarene derivative monolayers such as example 7 are also prepared according to the same method.

### EXAMPLE 11

Preparation of an Iminecalixarene Derivative Monolayer on a Gold Substrate as Shown in FIG. 4

A solution, wherein TMBICAT synthesized in example 5 is dissolved in an organic solvent such as CHCl₃, etc. in a concentration of 0.1-5 mM, is prepared. As shown in FIG. 4, a gold substrate is put in the prepared solution for 1˜24 hours, and then taken out and washed with chloroform, acetone and water each, and dried to prepare the iminecalixarene monolayer shown in FIG. 4. Other iminecalixarene derivative monolayers are also prepared according to the same method. Said gold substrate can be used in various forms, but in general, a substrate vacuum-plated with gold in a thickness of 100-300 nm after vacuum-plating glass, fused silica, silicon wafer, plastic substrate, etc. with chromium (Cr) or titanium (Ti), etc. in a thickness of 5-20 nm, is used. The thus-prepared gold substrate is put in a piranha solution (a mixed solution in the ratio of strong sulfuric acid:30% hydrogen peroxide=3:1) for 10 seconds˜1 minute just before use, and then washed with water and dried under nitrogen exchange, and used immediately. The formation of the monolayer is analyzed using infrared external reflection spectroscopy (FTIR-ERS).

The monolayer of other iminecalixarene derivatives as shown in example 9 is also prepared by the same method.

### EXAMPLE 12

Preparation of the Aminocalixarene Derivative Monolayer of FIG. 3

A solution, wherein a derivative having an aldehyde terminal among aminocalixarene derivatives of formula 6 or formula 7 is dissolved in an organic solvent such as CHCl₃, etc. in a concentration of 0.1˜5 mM, is prepared. A slide glass (amine chip) wherein an amine functional group is attached, prepared as shown in FIG. 1, is put in the prepared solution for 1˜24 hours, and then taken out and washed with chloroform, acetone and water each, and dried to prepare the aminocalixarene monolayer shown in FIG. 3.

### EXAMPLE 13

General Unmodified Protein Fixation Method by Molecular Recognition Shown in FIG. 6

Fixation of Antibody Protein

The sample solutions used to immobilize protein on the iminecalixarene derivative monolayer prepared in example 1, concentrations, compositions, etc. are as shown table 1. First, for the fixation of protein, a well plastic having a hole with a diameter of 2 mm is attached onto the chip. On the chip wherein the well plastic is attached, a protein sample solution prepared as shown in table 1 is coated in 3 ul each using a micropipet, and it is fixed in a plastic chamber for 1 hour at room temperature. At this time, the amount of coated antibody is three times as much as can cover the surface once. After fixation, in order to block the positions other than the spots wherein protein is fixed, it is put in a blocking solution. After the well plastic is detached from the chip, the chip is left in the blocking solution for 30 minutes at room temperature. After blocking, in order to clean the chip, it is put in a cleaning solution 1 for 2 minutes, and a cleaning solution 2 for 2 minutes, and then taken out and dried.

Reaction with Antibody Protein

After being dried, the chip wherein antibody is fixed is put in 15 ml of the prepared antigen solution in order to react with fluorescent attached antigen, and incubated in a chamber for 1 hour at 37° C. After the antigen-antibody reaction is completed, the chip is put in cleaning solution 1 for 2 minutes, and in cleaning solution 2 for 2 minutes. Then it is dried and analyzed using a fluorescence scanner (GSI Lumonics, U.S.A.).

TABLE 1

| | |
|---|---|
| 1 × PBS buffer | NaCl 8 g, KCl 0.2 g, Na₂HPO₄ 1.44 g, KH₂PO₄ 0.24 g in DW 1000 ml |
| Protein (antibody) | (90%) glycerol 14 μl + CRP antigen (300 μg/μl)7.5 μl + |
| solution 1 | 1 × PBS 28.5 μl = 50 μl |
| Fluorescent | 5% milk 20 μl + CRP antigen (300 μl/ml)50 μl + 1 × |
| attached protein | PBS 930 μl = 1.0 ml |
| (antibody) solution 2 | |
| Blocking solution | non-fat powdered milk 2.5 g + 1 x PBS 50 ml = 50 ml |
| Cleaning solution 1 | Tween20 300 μl + 1 x PBS 1000 ml = 1000 ml |
| Cleaning solution 2 | 1 × PBS composition |

### EXAMPLE 14

Preparation of the Aminocalixarene Derivative Monolayer on a Gold Substrate of FIG. 5

A solution, wherein a derivative having a thiol terminal among aminocalixarene derivatives of formula 6 or formula 7 is dissolved in an organic solvent such as CHCl₃, etc. in a concentration of 0.1-5 mM, is prepared. As shown in FIG. 5, a gold substrate is put in the prepared solution for 1˜24 hours, and then taken out and washed with chloroform, acetone and water each, and dried to prepare the aminocalixarene monolayer shown in FIG. 5. Said gold substrate can be used in various forms, but in general, a substrate vacuum-plated with gold in a thickness of 100-300 nm after vacuum-plating glass, fused silica, silicon wafer, plastic substrate, etc. with chromium (Cr) or titanium (Ti), etc. in a thickness of 5-20 nm, is used. The thus-prepared gold substrate is put in a piranha solution (a mixed solution in the ratio of strong sulfuric acid:30% hydrogen peroxide=3:1) for 10 seconds˜1 minute just before use, and then washed with water and dried under nitrogen exchange, and used immediately. The formation of the monolayer is analyzed using infrared external reflection spectroscopy (FTIR-ERS).

### EXAMPLE 15

Ion Concentration Effect Upon Protein Fixation

The sample solutions used for the protein fixation involving ions on the iminecalixarene derivative monolayer, their concentration and composition, etc. are as shown in Table 2 or 4. First, for the fixation of protein, well plastic with a hole having a diameter of 2 mm is attached onto the chip. On the chip wherein well plastic is attached, a protein sample solution prepared as shown in table 2 or 4 is coated using a micropipet in 3 ul for each ion and concentration. Then, it is fixed in a plastic chamber for 10 minutes, 30 minutes, and 1 hour for each ion. After fixation, in order to block the positions other than the spots wherein protein is fixed, the chip is put in a blocking solution of table 1, and the well plastic is detached from the chip, and then the chip is left in the blocking solution for 30 minutes at room temperature. After blocking, the chip is put in cleaning solution 1 of table 1 for 2 minutes, and cleaning solution 2 of table 1 for 2 minutes for cleaning, and then taken out and dried.

Reaction of the Fixed Antibody and Antigen

After being dried, the chip wherein antibody is fixed as a monolayer is put in 15 my of fluorescent attached protein (antibody) solution 2 of table 1, and incubated in a chamber for 1 hour at 37° C. in order to react with fluorescent attached antigen. After antigen-antibody reaction is completed, the chip is put in cleaning solution 1 of table 1 for 2 minutes, and cleaning solution 2 of table 1 for 2 minutes, and then dried. The analysis result using a fluorescence scanner (GSI Lumonics, U.S.A.) is shown in FIG. 4. According to the result of FIG. 4, if fixation proceeds using Na⁺, K⁺, at an ion concentration of 100 mM or more, the maximum fluorescence is achieved at 1 hour. The fixation density of the antibody protein combined with fluorescent attached antigen at 30 minutes is at the level of 80% of the maximum, and it increases to the level of 90% at 1 hour. On the other hand, it is shown that if NH4⁺ ion is used, at the level of an ion concentration of 100 mM˜800 mM, about 90% of the maximum fixation density is achieved by 10-minute fixation and it reaches almost 100% at 30 minutes. In light of the phenomenon that protein in a solution phase loses activity as time passes by, compared with a conventional fixation technology according to which it takes about several hours to 1 day to immobilize protein while maintaining the maximum activity, in the case of fixation on the iminecalixarene derivative chip, products can be prepared faster than in the conventional technology since superhigh speed fixation wherein protein is fixed in about 30 minutes at a superhigh speed is possible, and at the same time, the protein activity is maintained relatively more reproducibly than in the fixation by conventional technology and the same results are obtained throughout several repeated experiments since the time that protein exists in a liquid solution decreases from more than several hours to the level of several minutes.

TABLE 2

| | | | 1 × | | |
|---|---|---|---|---|---|
| | | | PBS in | | |
| | Na⁺ | (90%) | CRP Ab | | Gross |
| Concentration | concentration | glycerol | 300 μg/ml | DW | volume |
| 0 mM | 0 μl | 14 μl | 7.5 μl | 28.5 μl | 50 μl |
| 25 mM | (0.5M)2.5 μl | 14 μl | 7.5 μl | 26 μl | 50 μl |
| 50 mM | (0.5M)5 μl | 14 μl | 7.5 μl | 23.5 μl | 50 μl |
| 100 mM | (0.5M)10 μl | 14 μl | 7.5 μl | 18.5 μl | 50 μl |
| 200 mM | (0.5M)20 μl | 14 μl | 7.5 μl | 8.5 μl | 50 μl |
| 400 mM | (4M)5 μl | 14 μl | 7.5 μl | 23.5 μl | 50 μl |
| 800 mM | (4M)10 μl | 14 μl | 7.5 μl | 18.5 μl | 50 μl |

TABLE 3

| | | | 1 × | | |
|---|---|---|---|---|---|
| | | | PBS in | | |
| | K⁺ | (90%) | CRP Ab | | Gross |
| Concentration | concentration | glycerol | 300 μg/ml | DW | volume |
| 0 mM | 0 μl | 14 μl | 7.5 μl | 28.5 μl | 50 μl |
| 25 mM | (0.5M)2.5 μl | 14 μl | 7.5 μl | 26 μl | 50 μl |
| 50 mM | (0.5M)5 μl | 14 μl | 7.5 μl | 23.5 μl | 50 μl |
| 100 mM | (0.5M)10 μl | 14 μl | 7.5 μl | 18.5 μl | 50 μl |
| 200 mM | (0.5M)20 μl | 14 μl | 7.5 μl | 8.5 μl | 50 μl |
| 400 mM | (4M)5 μl | 14 μl | 7.5 μl | 23.5 μl | 50 μl |
| 800 mM | (4M)10 μl | 14 μl | 7.5 μl | 18.5 μl | 50 μl |

TABLE 4

| | | | 1 × | | |
|---|---|---|---|---|---|
| | | | PBS in | | |
| | NH₄⁺ | (90%) | CRP Ab | | Gross |
| Concentration | concentration | glycerol | 300 μg/ml | DW | volume |
| 0 mM | 0 μl | 14 μl | 7.5 μl | 28.5 μl | 50 μl |
| 25 mM | (0.5M)2.5 μl | 14 μl | 7.5 μl | 26 μl | 50 μl |
| 50 mM | (0.5M)5 μl | 14 μl | 7.5 μl | 23.5 μl | 50 μl |
| 100 mM | (0.5M)10 μl | 14 μl | 7.5 μl | 18.5 μl | 50 μl |
| 200 mM | (0.5M)20 μl | 14 μl | 7.5 μl | 8.5 μl | 50 μl |
| 400 mM | (4M)5 μl | 14 μl | 7.5 μl | 23.5 μl | 50 μl |
| 800 mM | (4M)10 μl | 14 μl | 7.5 μl | 18.5 μl | 50 μl |

### EXAMPLE 16

The Effect of an Ammonium Ion Concentration Upon the Fixation of Unmodified Protein by Molecular Recognition of FIG. 7

The Fixation of Antibody Proteins Containing Ammonium Ions in Different Concentrations

The concentrations, compositions, etc. of the cleaning solutions and the blocking solution used to immobilize proteins on the aminocalixarene derivative monolayer prepared in example 12, i.e. aminocalixarene derivative chip is as shown in Table 5. The fixation solutions containing ammonium ions in different concentrations as an fixation solution used for fixation of antibody proteins and the reaction solution used for the reaction of fluorescent antigen are as shown in FIG. 6. First, for the fixation of protein, well plastic having a hole with a diameter of 2 mm is attached onto the chip. After CRP antibody protein of the same concentration (the concentration 3 times higher than the theoretical maximum fixation value, 135 ng/0.0314 cm²) and fixation solutions 1, 2, 3, 4, 5, 6, and 7 of Table 6 containing ammonium ions of different concentrations each are coated on the chips in 3 μl each using a micropipet, they are fixed in a constant temperature oven wherein the humidity is maintained for 10 minutes, 30 minutes, and 1 hour for each at room temperature. The density of protein fixed in the fixation of more than 1 hour did not show much difference from the result of that of 1 hour. In order to block the positions other than the spots wherein antibody protein is coated and fixed, the chips are put in a blocking solution of table 1. At this time, after the well plastic is detached from the chip, the chips are left in the blocking solution for 30 minutes at room temperature. After blocking, in order to clean the chips, they are put in cleaning solution 1 of table 5 for 2 minutes, and cleaning solution 2 of table 5 for 2 minutes, and then taken out and dried.

The Reaction with Fluorescent Attached Protein

The method for reacting the fluorescent CRP antigen protein to be combined with the antibody of the fixed antibody protein monolayer on the aminocalixarene derivative monolayer is as follows. After the aminocalixarene derivative chip wherein protein is fixed in 15 ml of fluorescent antigen solution (antigen concentration 1.4 g/ml) presented in Table 6, combination reaction was carried out maintaining 37° C. for 1 hour. After antigen-antibody reaction is completed, the chip is put in cleaning solution 1 of Table 5 for 2 minutes, and then cleaning solution 2 for 2 minutes, and dried. Then, it is analyzed using fluorescence scanner (GSI Lumonics, U.S.A.). The result is as shown in FIG. 6. According to the result of FIG. 10, in a fixation in the fixation solution wherein the ammonium ion (NH₄⁺) concentration is as low as about 0˜100 mM, in the case of fixation for 1 hour, the protein fixation density is at the level of 40% of the theoretical maximum value, showing a low fixation density. On the other hand, in case the ammonium ion (NH₄⁺) concentration is about 400 mM, the fixation density at the level of 90% of the theoretical maximum value is achieved, and also, fixation time-wise, protein fixation has proceeded at very high density in a short time of about 30 minutes to 1 hour. In case the ammonium ion (NH₄⁺) concentration is 800 mM, since the ion concentration is too high, the active site of antibody protein goes through alteration as well-known, causing activity reduction. In this case, although protein fixation proceeds at the maximum density as in the case of 400 mM, since the activity of the fixed antibody is lowered, combination reaction with fluorescent attached antigen proceeds at a lower level than in the case of using a concentration of 400 mM, and thus, fluorescent attached antigen which is actually attached is at the level of 50% of the theoretical maximum value.

TABLE 5
Cleaning solution and blocking solution

| | |
|---|---|
| 1 × PBS buffer | (NaCl 8 g, KCl 0.2 g, Na₂HPO₄ 1.44 g, KH₂PO₄ |
| | 0.24 g)/DW 1000 ml |
| Blocking solution | non-fat powdered milk 2.5 g + 1 × PBS 50 ml = |
| | 50 ml |
| Cleaning solution 1 | Tween20 300 μl + 1 × PBS 1000 ml = 1000 ml |
| Cleaning solution 2 | (NaCl 8 g, KCl 0.2 g, Na₂HPO₄ 1.44 g, KH₂PO₄ |
| | 0.24 g)/DW 1000 ml |

TABLE 6
Fixation solution and fluorescent attached antigen solution

| | NH₄⁺ concentration | | | | | |
|---|---|---|---|---|---|---|
| | and the added amount | | (90%) | CRP Ab | Purified | Gross |
| | of NH₄⁺Cl | | glycerol | 300 μg/ml | water(DW) | volume |
| Fixation | 0 mM | 0 μl | 14 μl | 7.5 μl | 28.5 μl | 50 μl |
| solution 1 | | | | | | |
| Fixation | 25 mM | (0.5M)2.5 μl | 14 μl | 7.5 μl | 26 μl | 50 μl |
| solution 2 | | | | | | |
| Fixation | 50 mM | (0.5M)5 μl | 14 μl | 7.5 μl | 23.5 μl | 50 μl |
| solution 3 | | | | | | |
| Fixation | 100 mM | (0.5M)10 μl | 14 μl | 7.5 μl | 18.5 μl | 50 μl |
| solution 4 | | | | | | |
| Fixation | 200 mM | (0.5M)20 μl | 14 μl | 7.5 μl | 8.5 μl | 50 μl |
| solution 5 | | | | | | |
| Fixation | 400 mM | (4M)5 μl | 14 μl | 7.5 μl | 23.5 μl | 50 μl |
| solution 6 | | | | | | |
| Fixation | 800 mM | (4M)10 μl | 14 μl | 7.5 μl | 18.5 μl | 50 μl |
| solution 7 | | | | | | |

| | |
|---|---|
| Fluorescent | antigen concentration 1.4 μg/ml = (5% milk 300 μl + Cy5-CRP |
| attached | antigen(818 μg/ml)27 μl + 1 × PBS 14.7 ml = 15 ml) |
| antigen | |
| solution | |

### EXAMPLE 17

Fluorescence Analysis Technology for the Analysis of Protein Fixation Maximum Density

The result shown in FIG. 11 is the result obtained by coating a glass slide with fluorescent attached antigen in the amount 0.5, 1, 2 times that of the fluorescent attached antigen needed (1.1 μg/cm²) to form 1:1 combination with 1.4 μg/cm² of antibody (CRP Ab 160 kD) needed to cover the area of 1 cm² on the surface of aminocalixarene derivative monolayer one time, and drying it, and then analyzing the fluorescence sensitivity with fluorescence scanner (GSI Lumonics, U.S.A.) directly. If 1 μl of fluorescent attached antigen is coated, a circular spot having a diameter 2 mm is created, and its dimension is 0.0314 cm². Thus, it corresponds to coating the amount, 3(5 μg/ml)×1 ul/0.0314 cm²=1.1 μg/cm², of fluorescent attached antigen which can form 1:1 combination with the fixed antibody on the coated space. The result presented in FIG. 11 could be obtained by carrying out the present example using the same antigen as that used for combination with the fixed antibody. It is shown that the fluorescence sensitivity when 0.5 time as much is coated is at the level of 55% of that of in case when 30 minutes of fixation has proceeded using ammonium ion as shown in FIG. 9, and in case of the same amount being coated, the fluorescence sensitivity is almost at the same level as in case when 30 minutes of fixation has proceeded using ammonium ion, and the product is reacted with the same fluorescent attached antigen, and the fluorescence sensitivity is analyzed as shown in FIG. 4. Thus, this result shows that the antibody protein fixation density achieved in the protein fixation which has proceeded using ammonium ion in FIG. 9 reaches the theoretical maximum value, that is, a complete monolayer is prepared, and at the same time the activity is excellent enough to form 1:1 combination with antigen.

### EXAMPLE 18

Competitive Fixation with an Amine Functional Group Upon Protein Fixation

Competitive protein fixation with an amine functional group compound on the iminecalixarene chip shown in FIG. 12. diagram was carried out in the same method as the antibody protein fixation method of example 13, and the sample solutions, concentrations, compositions, etc. are as shown in Table 7. After 30 mM of alanine, phenylalanine, phenethylalanine, etc. are mixed, the fixation proceeds for 1 hour. Then, in the same method as the treatment method after antibody protein fixation of example 13, iminecalixarene derivative monolayer is washed, and blocking is carried out. Antibody-antigen reaction is carried out using the fluorescent attached protein (antigen) of Table 1 in the same method as in example 13, and treated in the same method. The result analyzed by using a fluorescence scanner (GSI Lumonics, U.S.A.) is presented in FIG. 14. In case of using alanine, the fixation speed is hardly affected; however, in case of using phenylalanine or phenethylamine, which is recognized fast by iminecalixarene, the fluorescence decreases sharply, showing that such amine compounds are recognized by the iminecalixarene derivative in competition with antibody protein, and as a result inhibit the fixation of antibody protein, which makes the fixation proceed at a very low density. This is the result showing that when protein fixation occurs on the iminecalxarene derivative monolayer, the protein is ultimately fixed while the parts with an amine functional group (ammonium group in a solution phase) being fixed irreversibly.

In addition, this is the result showing that on the iminecalixarene derivative monolayer developed in the present invention the protein fixation proceeds at a superhigh speed by ionic/molecular recognition.

TABLE 7

| Prepared to | | | | | |
|---|---|---|---|---|---|
| make 30 mM | | | | | |
| of an amine | Added | (90%) | 1 × PBS in | 1 × | Gross |
| compound | amount | glycerol | CRP Ab 300 μg/ml | PBS | volume |
| Ala | (1.0M)3 μl | 28 μl | 15 μl | 54 μl | 100 μl |
| PBA | (1.0M)3 μl | 28 μl | 15 μl | 54 μl | 100 μl |
| Pha | (1.0M)3 μl | 28 μl | 15 μl | 54 μl | 100 μl |

### EXAMPLE 19

Measurement of Fixation Speed Reduction by a Competitive Reaction with an Ammonium Functional Group Upon Protein Fixation

Competitive protein fixation with an amine functional group compound on the aminocalixarene chip shown in FIG. 14. diagram was carried out in a method that the fixation is carried out for 1 hour using 400 mM of ammonium ion, in the same way as in the antibody protein fixation method involving ammonium ion of example 16. The concentration of the used sample solutions containing an amine functional group compound, their composition, etc. are as shown in Table 8. After the solution without an amine compound (control), competitive solutions 1, 2, 3, and 4 wherein 10 mM of aniline, benzylamine, lysine, etc. is mixed, are coated with antibody protein for fixation in 3 μl each, the fixation is carried out for 1 hour. Then, the aminocalixarene derivative monolayer is washed in the same way as in the treatment method after antibody protein fixation of example 16, and blocking is carried out. Then, antibody-antigen reaction is carried out using the fluorescent attached antigen solution of Table 6 in the same method as in the reaction with fluorescent attached antigen of example 16, and after it is treated in the same method, it is analyzed using a fluorescence scanner (GSI Lumonics, U.S.A.). The result is as shown in FIG. 15. Under protein fixation conditions wherein 10 mM of an amine compound is contained each, in case of using aniline, whose amine functional group cannot change to an ammonium functional group, it hardly affects the antibody protein fixation speed, and thus the antibody protein fixation density at the same level of that of the case that there is no amine compound was achieved. On the other hand, in case of using benzylamine or lysine, which exists as an ammonium group under the experiment condition, fluorescence decreased sharply, which is because amine compounds are ionic/molecular recognized by the aminocalixarene derivative in competition with antibody protein, and as a result the antibody protein fixation is inhibited and thus the fixation is achieved at a low density. In addition, this is the result proving that protein fixation on the aminocalixarene derivative monolayer proceeds as the parts having an amine functional group (ammonium group in a liquid solution) are fixed through irreversible ionic/molecular recognition.

TABLE 8
Fixation solution containing 10 mM of an amine functional group
compound

| | | The | | | | | |
|---|---|---|---|---|---|---|---|
| | | amount | | | | | |
| | 10 mM of | of an | | | | | |
| | an amine | added | | | | | |
| | compound | amine | (90%) | CRP Ab | Purified | | Gross |
| | contained | compound | glycerol | 300 μg/ml | water(DW) | 4N)NH₄⁺Cl | volume |
| Competitive | Control | 0 μl | 28 μl | 15 μl | 47 μl | 10 μl | 100 μl |
| solution 1 | | | | | | | |
| Competitive | Aniline | (100 mM) | 28 μl | 15 μl | 37 μl | 10 μl | 100 μl |
| solution 2 | | 10 μl | | | | | |
| Competitive | Benzylamine | (100 mM) | 28 μl | 15 μl | 37 μl | 10 μl | 100 μl |
| solution 3 | | 10 μl | | | | | |
| Competitive | Lysine | (100 mM) | 28 μl | 15 μl | 37 μl | 10 μl | 100 μl |
| solution 4 | | 10 μl | | | | | |

In addition, the protein fixation on the aminocalixarene derivative presented in the present invention is a technology of fixing unmodified proteins, which prepares a protein chip by performing fixation on the surface of a monolayer in a short time of about 30 minutes to 1 hour at a theoretically maximum density, that is, at a superhigh speed and a high density by molecular/ionic recognition.

### EXAMPLE 20

Conjugation Reaction of Cy5 (Fluorescent Material) and Antigen of FIG. 16 to Attach Fluorescence to Antibody Protein

Fluorescent attached antigen protein is prepared by reacting Cy-5, a fluorescent material provided by Telechem (U.S.A), as follows according to the provided condition for conjugation reaction with the protein.

325 μl of Cy-5 fluorescent material diluted in a carbonate buffer is put in a tube. After 175 μl of CRP antibody protein (2.86 mg/ml) diluted in a 1×PBS solution is put in said tube, it is reacted for 30 minutes at room temperature. After the reaction, the reaction product is column-refined (Sephadex™ G-50), and by analyzing the absorption band ratio of the amide functional group of protein and the aromatic group of the fluorescent material according to the analysis method provided by Telechem, the number of fluorescence attached to each protein is measured. After it is confirmed that about 1 fluorescence is attached to each protein, it is used for the combination reaction with antibody protein. After the amount of antigen protein combined with the fixed antibody protein is measured using a fluorescence scanner (GSI Lumonics, U.S.A.), density of the fixed protein is confirmed through fluorescence analysis.

### EXAMPLE 21

Analysis of Iminecalixarene Derivative, TBICAP (5,11,17,23-tetrabenzyliminecalix[4]arene-1,3-dipropyl) Used for NMR Analysis of the Iminecalixarene Derivative and the Amine Compound in FIG. 18 and FIG. 19

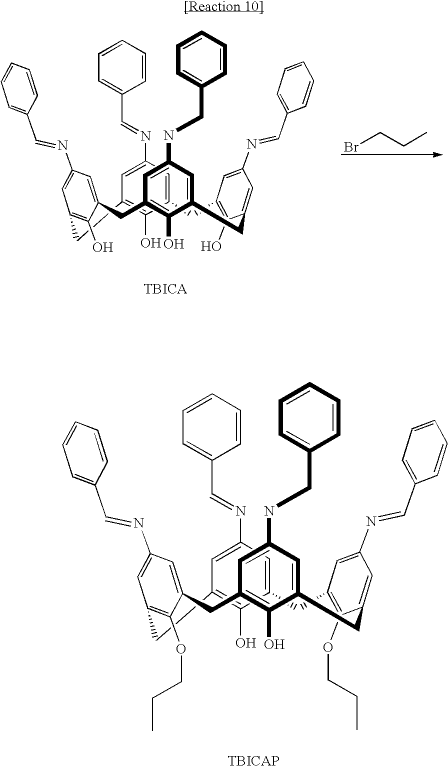

After TBICA (500 mg, 0.59 mmol), anhydrous K₂CO₃ (826 mg, 5.9 mmol) and sodium iodide (1.3 g, 8.9 mmol) are put in a dried round-bottom flask, it is dried for 30 minutes under decompressed pressure. Then, after 120 ml of anhydrous acetonitrile is added under nitrogen exchange, it is mixed on a heater. After 10 minutes, 1-bromobutane (0.51 ml, 5.9 mmol) is added to the reaction vessel, and it is mixed for 24 hours with raising the temperature of the reaction vessel to 80° C. gradually. After the reaction, the solvent is decompressed and removed, and the remaining product is dissolved with CH₂Cl₂. Solid precipitates obtained during the reaction, such as KBr, KI, K₂CO₃ are removed by being decompressed and filtered and the filtered solution is decompressed to remove the solvent. The liquid product remaining in the flask is dissolved in 2 ml of CH₂Cl₂, and 13 ml of n-hexane is added slowly to extract the product. The extracted solid product is dissolved in 2 ml of CH₂Cl₂ once again, and 13 ml of n-hexane is added slowly to obtain 495 mg (yield 90%) of a yellow solid product, TBICAP (5,11,17,23-tetrabenzyliminecalix[4]arene-1,3-dipropyl)

¹H NMR (300 MHz CDCl₃): 8.47(s, 2H, N═CH), 8.27(s, 2H, N═CH), 7.86-7.72(m, 8H, ArH), 7.42-7.33(m, 12H, ArH), 7.09(s, 4H, ArH), 6.86(s, 4H, ArH), 4.37(d, 4H, ArCH₂Ar), 4.02(t, 4H, OCH₂), 3.46(d, 4H, ArCH₂Ar), 2.10(q, 4H, OCH₂CH₂), 1.35(t, 6H, OCH₂CH₂CH₃)

### EXAMPLE 22

The Research on Molecular Recognition Between Allylamine and TBICAP by Using NMR

13.8 mg (920 g/mol, 0.015 mol) of TBICAP synthesized in example 21 is put in a vial, and dissolved in 5 ml of DMSO to make 3 mM. Then, after 500 μl of this TBICAP solution is put in an NMR tube, 2.5 μl of trimethylamine is added to the tube to make the solution under the basic condition. Allylamine is dissolved in D₂O₂ to a concentration of 500 mM, and 50 μl is taken and put in the prepared NMR tube, and then NMR is measured with molecular recognition proceeding, the result of which is shown in FIG. 19. (TBICAP:allylamine=1:16) The fact that the iminecalixarene is irreversibly combined with allylamine, an amine compound, is proven by the result that imine hydrogens, a, a′ which appears around 8.6 ppm, appeared around 8.3 as a single peak. It is also found that calixarene is irreversibly combined with allylamine by molecular recognition from the result that 8 hydrogens of calixarene, which appears around 7.2 ppm, moved to 6.2 ppm by nearly 1 ppm. In addition, the number of recognized allylamines can be grasped from CH₂—NH₂, i.e. the hydrogens attached to the carbon next to amine, and it was confirmed that some of the hydrogens, which generally appears at 3.4 ppm, appeared at 4.2 ppm and integral value was shown as 8 hydrogens, showing that 4 allylamines are irreversibly molecular recognized by the iminecalixarene derivative. In addition, since the hydrogen of the allylamine appears at 3.4 ppm, moving from 3.2 ppm, when amine changes to ammonium, it is found that the allylamine which was molecular-recognized in this NMR tube was molecular/ionic-recognized in a state of allylammonium. The NMR result showing that some of the other hydrogens of allylamine has also moved, that is, only 4 times out of 16 times of used allylamine has moved, is shown in the NMR analysis of FIG. 19. Such results show that in the iminecalixarene derivative 4 nitrogens at the imine site and 4 ammonium ions are ionic/molecular recognized, which is illustrated in FIG. 18.

### EXAMPLE 23

The Research on Molecular Recognition of TBICAP with Alanine, Phenylalanine, Phenethylamine, etc. Using NMR

TBICAP material (13.8 mg, 0.015 mmol) synthesized in example 21 is measured and put in a vial, and dissolved in 5 ml of DMSO to make 3 mM. Then, after each amine compound is prepared in the same way as in example 21, it is added to NMR tube in the ratio of 1:16 as in example 22. By obtaining NMR spectrum by the hour, time needed for molecular recognition to proceed completely is grasped, leading to the analysis of a compound that allows an easy, or strong, molecular recognition. According to the result shown in FIG. 18, the compound having a benzene ring shows the NMR of the iminecalixarene derivative wherein molecular recognition has been completed within 2 hours, whereas in case of alanine, molecular recognition proceeds very slowly and as late as after 4 days, most of the molecular reaction is carried out. This result shows that if there is p-p stacking interaction between the benzene ring of the amine compound to be molecular recognized and the benzene ring of benzlylimine of the iminecalixarene derivative, molecular recognition proceeds faster. In addition, this result is identical with the result of FIG. 14 that shows in the competitive reaction between protein fixation and amine compounds, the amine compound wherein benzene ring is attached is strongly molecular/ionic recognized by the iminecalixarene derivative, inhibiting the protein fixation effectively. In addition, the iminecalixarene derivative monolayer developed in the present invention is the world s first superhigh speed unmodified protein fixation technology that can prepare a protein chip by fixing protein on the surface of a monolayer in a short time at a superhigh speed without any alteration.

### INDUSTRIAL APPLICABILITY

Distinguished completely from the conventional method of preparing protein chip by fixing protein using conventional chemical bonding, physical absorption, or biotin-streptavidin, which is being generally used over the world, the present invention is a novel technology wherein once unmodified protein is coated on an imincalixarene derivative monolayer or an aminocalixarene derivative monolayer, protein is fixed on the surface of the monolayer irreversibly at superhigh speed through ionic/molecular recognition and at the same time with the maximum density fixation.

1-19. (canceled)

20. An iminecalixarene derivative of Formula 2: 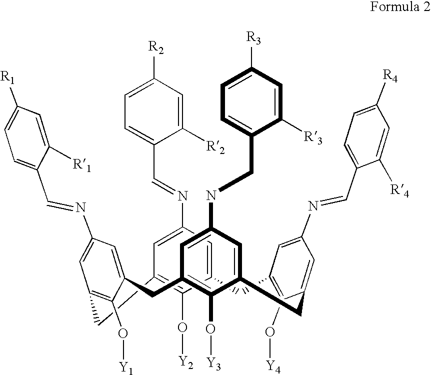
wherein R₁, R′₁, R₂, R′₂, R₃, R′₃, R₄, and R′₄ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H_{5,} —OC₆H₅, —OC₆H₄CH₃, —OC₆H₄C(CH₃)₃, —OC₆H₄CF₃, —OC₆H₄Cl, —OCOCH₃, —NHCOCH₃, —CONHCH₃, —CN, COOH, —COOCH₃ and —COOC₂H₅;
Y₁, Y₂, Y₃ and Y₄ are independently selected from the group consisting of —H, —(CH₂)ₙ—CH═O, —(CH₂)ₙ—SH, —(CH₂CH₂O)ₘ—CH₂CH₂—CH═O, —(CH₂CH₂O)ₘ—CH₂CH₂—SH, —(CH₂)ₘ—C₆H₄—(CH₂)_{c}-Z and —CO—(CH₂)ₘ₋₁—C₆H₄—(CH₂)_{c}-Z, where n=2-15, m=1-10, c=0-10, Z=—SH, —CHO, —COOH or —NH₂, and —C₆H₄ and —C₆H₅ are defined as a phenyl group.

21. The iminecalixarene derivative according to claim 20, wherein Y₁, Y₂, Y₃ and Y₄ are H.

22. An iminecalixarene derivative of Formula 4: 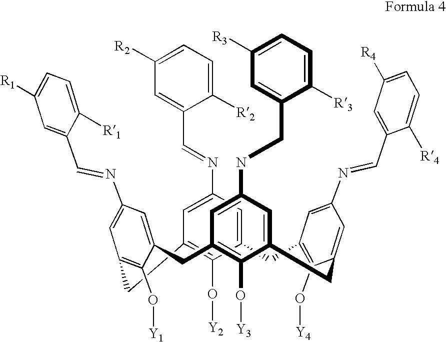
wherein, R₁, R′₁, R₂, R′₂, R₃, R′₃, R₄, and R′₄ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H₅, —OC₆H₅, —OC₆H₄CH₃, —OC₆H₄C(CH₃)₃, —OC₆H₄CF₃, —OC₆H₄Cl, —OCOCH₃, —NHCOCH₃, —CONHCH₃, —CN, COOH, —COOCH₃ and —COOC₂H₅;
Y₁, Y₂, Y₃ and Y₄ are independently selected from the group consisting of —H, —(CH₂)ₙ—CH═O, —(CH₂)ₙ—SH, —(CH₂CH₂O)ₘ—CH₂CH₂—CH═O, —(CH₂CH₂O)ₘ—CH₂CH₂—SH, —(CH₂)ₘ—C₆H₄—(CH₂)_{c}-Z and —CO—(CH₂)ₘ₋₁—C₆H₄—(CH₂)_{c}-Z, where n=2-15, m=1-10, c=0-10, Z=—SH, —CHO, —COOH or —NH₂, and —C₆H₄ and —C₆H₅ are defined as phenyl group.

23. The iminecalixarene derivative according to claim 22, wherein Y₁, Y₂, Y₃ and Y₄ are H.

24. A method for preparing the iminecalixarene derivative according to claim 21, comprising the step of imine-bonding a compound of Formula 5 and a benzealdehyde derivative:

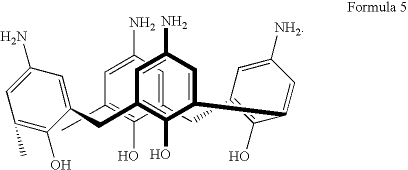

25. A method for preparing the iminecalixarene derivative according to claim 23, comprising the step of imine-bonding a compound of Formula 5 and a benzealdehyde derivative:

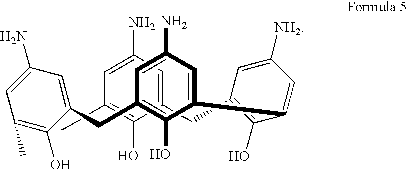

26. An iminecalixarene derivative monolayer solid substrate prepared by imine-bonding the iminecalixarene derivative according to claim 20, having an aldehyde terminal, in solution to a solid substrate selected from the group consisting of glass, siliconwafer and crystal, having an amine group.

27. An iminecalixarene derivative monolayer solid substrate prepared by imine-bonding the iminecalixarene derivative according to claim 22, having an aldehyde terminal, in solution to a solid substrate selected from the group consisting of glass, siliconwafer and crystal, having an amine group.

28. An iminecalixarene derivative monolayer solid substrate prepared by ester-, ether-, amine-, or amide-bonding the iminecalixarene derivative according to claim 20 in solution to a solid substrate selected from the group consisting of glass, siliconwafer and crystal, through a functional group of the iminecalixarene derivative.

29. An iminecalixarene derivative monolayer solid substrate according to claim 28, wherein said solid substrate is gold and said functional group is a thiol group.

30. An iminecalixarene derivative monolayer solid substrate prepared by ester-, ether-, amine-, or amide-bonding the iminecalixarene derivative according to claim 22 in solution to a solid substrate selected from the group consisting of glass, siliconwafer and crystal, through a functional group of the iminecalixarene derivative.

31. An iminecalixarene derivative monolayer solid substrate according to claim 30, wherein said solid substrate is gold and said functional group is a thiol group.

32. A protein chip prepared by fixing a protein through irreversible molecule or ion recognization on the iminecalixarene derivative monolayer solid substrate according to claim 26.

33. A protein chip prepared by fixing a protein through irreversible molecule or ion recognization on the iminecalixarene derivative monolayer solid substrate according to claim 27.

34. A protein chip prepared by fixing a protein through irreversible molecule or ion recognization on the iminecalixarene derivative monolayer solid substrate according to claim 28.

35. A protein chip prepared by fixing a protein through irreversible molecule or ion recognization on the iminecalixarene derivative monolayer solid substrate according to claim 30.

36. An aminocalixarene derivative monolayer prepared by attaching an aminocalixarene derivative of Formula 6 or 7 to a solid substrate: 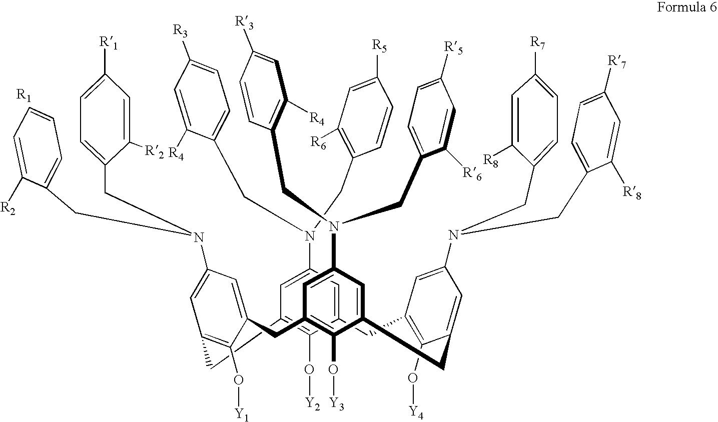
wherein, R₁, R₂, R₃, R₄, R₅, R₆, R₇, R₈, R′₁, R′₂, R′₃, R′₄, R′₅, R′₆, R′₇ and R′₈ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H₅, —OC₆H₅, —OC₆H₄CH₃, —OC₆H₄CF₃, —OC₆H₄CF₃, —OC₆H₄Cl, —OCOCH₃, —NHCOCH₃, —CONHCH₃, —CN, COOH, and —COOR, where R is —CH₃ or —C₂H₅;
Y₁, Y₂, Y₃ and Y₄ are independently selected from the group consisting of —H, —(CH₂)ₙ—CH═O, —(CH₂)ₙ—SH, —(CH₂CH₂O)ₘ—CH₂CH₂—CH═O, —(CH₂CH₂O)ₘ—CH₂CH₂—SH, —(CH₂)ₘ—C₆H₄—(CH₂)_{c}-Z and —CO—(CH₂)ₘ₋₁—C₆H₄—(CH₂)_{c}-Z, where n=2-15, m=1-10, c=0-10, Z=—SH, —CHO, —COOH or —NH₂, and —C₆H₄ and —C₆H₅ are defined as a phenyl group; 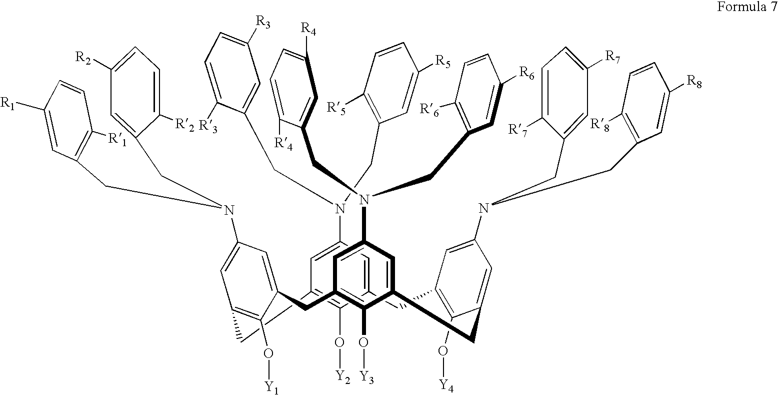
wherein, R₁, R₂, R₃, R₄, R₅, R₆, R₇, R₈, R′₁, R′₂, R′₃, R′₄, R′₅, R′₆, R′₇ and R′₈ are independently selected from the group consisting of —H, —CH₃, —C₂H₅, —C₃H₇, —OCH₃, —Cl, —C₆H₅, —OH, —OCH₂CH₃, —Br, —CF₃, —OCH₂C₆H₅, —OC₆H₅, —OC₆H₄CH₃, —OC₆H₄C(CH₃)₃, —OC₆H₄CF₃, —OC₆H₄Cl, —OCOCH₃, —NHCOCH₃, —CONHCH₃, —CN, COOH, and —COOR, where R is —CH₃ or —C₂H₅;
Y₁, Y₂, Y₃ and Y₄ are independently selected from the group consisting of —H, —(CH₂), —CH═O, —(CH₂)ₙ—SH, —(CH₂CH₂O)ₘ—CH₂CH₂—CH═O, —(CH₂CH₂O)ₘ—CH₂CH₂—SH, —(CH₂)ₘ—C₆H₄—(CH₂)_{c}-Z and —CO—(CH₂)ₘ₋₁—C₆H₄—(CH₂)_{c}-Z, where n=2-15, m=1-10, c=0-10, Z=—SH, —CHO, —COOH or —NH₂, and —C₆H₄ and —C₆H₅ are defined as a phenyl group.

37. The aminocalixarene derivative monolayer according to claim 36, prepared by attaching through imine-, amine-, amide-, urea-, urethane-, ester-, thioether-, thioester-, thiourethane-, ether-, or carbonate-bonding to a solid substrate selected from the group consisting of glass, siliconwafer or melted quartz having a functional group of amine, alcohol, thiol or carboxyl acid.

38. The aminocalixarene derivative monolayer according to claim 36, prepared by imine-, or amine-bonding an aldehyde functional group of the aminocalixarene derivative to an amine functional group of a solid substrate selected from the group consisting of glass, siliconwafer, and melted quartz.

39. The aminocalixarene derivative monolayer according to claim 36, prepared by attaching the aminocalixarene derivative having a thiol functional group to a gold substrate.

40. A protein chip prepared by fixing a protein to the aminocalixarene derivative monolayer of claim 36.

41. The protein chip according to claim 40, wherein the protein is an antibody.

42. The protein chip according to claim 40, prepared by voluntary protein fixation through irreversible molecule/ion recognization.

43. The protein chip according to claim 40, wherein the protein has a molecular weight of 10 kD or more.

44. The protein chip according to claim 42, prepared in a cation concentration of 10 mM to 800 mM, wherein the cation is NH₄⁺.
